SEQUENCE LISTING (82 sequences)

SEQ ID NO: 1 - DNA, 1137 bp, Artificial Sequence
  partial DNA sequence of plasmid pLoxCat27 comprising the LoxP-Cat-LoxP cassette
ctcggatccactagtaacggccgccagtgtgctggaattcgcccttggccgcataacttcgtatagtatacattatacgaagttatctagagttgcatgcctgcaggtccgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggactctagataacttcgtatagtatacattatacgaagttatgaagggcgaattctgcagatatccatcacact

SEQ ID NO: 2 - DNA, 61 bp, Artificial Sequence
  Primer ArcA1
cacattcttatcgttgaagacgagttggtaacacgcaacacgtgtaggctggagctgcttc

SEQ ID NO: 3 - DNA, 62 bp, Artificial Sequence
  Primer ArcA2
ttccagatcaccgcagaagcgataaccttcaccgtgaatggtcatatgaatatcctccttag

SEQ ID NO: 4 - DNA, 24 bp, Artificial Sequence
  Primer ArcA3
agttggtaacacgcaacacgcaac

SEQ ID NO: 5 - DNA, 23 bp, Artificial Sequence
  Primer ArcA4
cgcagaagcgataaccttcaccg

SEQ ID NO: 6 - DNA, 1320 bp, Artificial Sequence
  Partial sequence of pLoxCat1 comprising the lox-Cat-loxP cassette
aagcttaaggtgcacggcccacgtggccactagtacttctcgaggtcgacggtatcgataagctggatccataacttcgtataatgtatgctatacgaagttatctagagtccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcggacctgcaggcatgcaactctagataacttcgtataatgtatgctatacgaagttatgcggccgccatatgcatcctaggcctattaatattccggagtatacgtagccggctaacgttctagcatgcgaaatttaaagcgctgatatcgatcgcgcgcagatctgtcatgatgatcattgcaattggatccatatatagggcccggggttataattacctcaggtcgacgtcccatggccattgaattcgtaa

SEQ ID NO: 7 - DNA, 61 bp, Artificial Sequence
  Primer GalA
tcggttttcacagttgttacatttcttttcagtaaagtctggatgcatatggcggccgcat

SEQ ID NO: 8 - DNA, 65 bp, Artificial Sequence
  Primer GalP2
catgatgccctccaatatggttattttttattgtgaattagtctgtttcctgtgtgaaattgtta

SEQ ID NO: 9 - DNA, 60 bp, Artificial Sequence
  Primer GlkA
acttagtttgcccagcttgcaaaaggcatcgctgcaattggatgcatatggcggccgcat

SEQ ID NO: 10 - DNA, 67 bp, Artificial Sequence
  Primer Glk2
cattcttcaactgctccgctaaagtcaaaataattctttctcgtctgtttcctgtgtgaaattgtta

SEQ ID NO: 11 - DNA, 1270 bp, Artificial Sequence
  LoxP-cat-loxP Trc cassette “insert”
ggatgcatatggcggccgcataacttcgtatagcatacattatacgaagttatctagagttgcatgcctgcaggtccgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggactctagataacttcgtatagcatacattatacgaagttatggatcatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagac

SEQ ID NO: 12 - DNA, 30 bp, Artificial Sequence
  Primer GalB1
actttggtcgtgaacatttcccgtgggaaa

SEQ ID NO: 13 - DNA, 28 bp, Artificial Sequence
  Primer GalC11
agaaagataagcaccgaggatcccgata

SEQ ID NO: 14 - DNA, 26 bp, Artificial Sequence
  Primer GlkB1
aacaggagtgccaaacagtgcgccga

SEQ ID NO: 15 - DNA, 30 bp, Artificial Sequence
  Primer GlkC11
ctattcggcgcaaaatcaacgtgaccgcct

SEQ ID NO: 16 - DNA, 99 bp, Artificial Sequence
  Primer edd1
atgaatccacaattgttacgcgtaacaaatcgaatcattgaacgttcgcgcgagactcgctctgcttatctcgcccggatttatcgataagctggatcc

SEQ ID NO: 17 - DNA, 98 bp, Artificial Sequence
  Primer edd2
ttaaaaagtgatacaggttgcgccctgttcggcaccggacagtttttcacgcaaggcgctgaataattcacgtcctgtcggatgcatatggcggccgc

SEQ ID NO: 18 - DNA, 22 bp, Artificial Sequence
  Primer edd3
taacatgatcttgcgcagattg

SEQ ID NO: 19 - DNA, 21 bp, Artificial Sequence
  Primer edd4
actgcacactcggtacgcaga

SEQ ID NO: 20 - DNA, 29 bp, Artificial Sequence
  CN1, encoding mutated trc promoter driving glk expression
ctgacaattaatcatccggctcgtataat

SEQ ID NO: 21 - DNA, 29 bp, Artificial Sequence
  CN2, encoding parent trc promoter
ttgacaattaatcatccggctcgtataat

SEQ ID NO: 22 - DNA, 25 bp, Artificial Sequence
  Primer gapA1
atgaccatctgaccatttgtgtcaa

SEQ ID NO: 23 - DNA, 25 bp, Artificial Sequence
  Primer gapA2
aatgcgctaacagcgtaaagtcgtg

SEQ ID NO: 24 - DNA, 35 bp, Artificial Sequence
  Primer gapA3
gatacctactttgatagtcacatattccaccagct

SEQ ID NO: 25 - DNA, 35 bp, Artificial Sequence
  Primer gapA4
agctggtggaatatgtgactatcaaagtaggtatc

SEQ ID NO: 26 - DNA, 35 bp, Artificial Sequence
  Primer gapA5
gatacctactttgatagtcaaatattccaccagct

SEQ ID NO: 27 - DNA, 35 bp, Artificial Sequence
  Primer gapA6
agctggtggaatatttgactatcaaagtaggtatc

SEQ ID NO: 28 - DNA, 42 bp, Artificial Sequence
  short 1.5 GI promoter
gcccttgactatgccacatcctgagcaaataattcaaccact

SEQ ID NO: 29 - DNA, 98 bp, Artificial Sequence
  Primer gapA-R1
agtcatatattccaccagctatttgttagtgaataaaagtggttgaattatttgctcaggatgtggcatagtcaagggcatatgaatatcctccttag

SEQ ID NO: 30 - DNA, 80 bp, Artificial Sequence
  Primer gapA-R2
gctcacattacgtgactgattctaacaaaacattaacaccaactggcaaaattttgtccgtgtaggctggagctgcttcg

SEQ ID NO: 31 - DNA, 42 bp, Artificial Sequence
  short 1.20 GI promoter
gcccttgacgatgccacatcctgagcaaataattcaaccact

SEQ ID NO: 32 - DNA, 42 bp, Artificial Sequence
  short 1.6 GI promoter
gcccttgacaatgccacatcctgagcaaataattcaaccact

SEQ ID NO: 33 - DNA, 24 bp, Artificial Sequence
  Primer gapA-R3
gtcgacaaacgctggtatacctca

SEQ ID NO: 34 - DNA, 98 bp, Artificial Sequence
  Primer gapA-R4
agtcatatattccaccagctatttgttagtgaataaaagtggttgaattatttgctcaggatgtggcatcgtcaagggcatatgaatatcctccttag

SEQ ID NO: 35 - DNA, 98 bp, Artificial Sequence
  Primer gapA-R5
agtcatatattccaccagctatttgttagtgaataaaagtggttgaattatttgctcaggatgtggcattgtcaagggcatatgaatatcctccttag

SEQ ID NO: 36 - DNA, 60 bp, Artificial Sequence
  Primer mgsA-1
gtacattatggaactgacgactcgcactttacctgcgcggtgtaggctggagctgcttcg

SEQ ID NO: 37 - DNA, 60 bp, Artificial Sequence
  Primer mgsA-2
cttcagacggtccgcgagataacgctgataatcggggatccatatgaatatcctccttag

SEQ ID NO: 38 - DNA, 22 bp, Artificial Sequence
  Primer mgsA-3
cttgaattgttggatggcgatg

SEQ ID NO: 39 - DNA, 21 bp, Artificial Sequence
  Primer mgsA-4
cgtcacgttattggatgagag

SEQ ID NO: 40 - DNA, 100 bp, Artificial Sequence
  Primer PppcF
cgattttttaacatttccataagttacgcttatttaaagcgtcgtgaatttaatgacgtaaattcctgctatttattcgtgtgtaggctggagctgcttc

SEQ ID NO: 41 - DNA, 100 bp, Artificial Sequence
  Primer PppcR
tcgcattggcgcgaatatgctcgggctttgcttttcgtcagtggttgaattatttgctcaggatgtggcattgtcaagggcatatgaatatcctccttag

SEQ ID NO: 42 - DNA, 30 bp, Artificial Sequence
  Primer SeqppcR 7
gcggaatattgttcgttcatattaccccag

SEQ ID NO: 43 - DNA, 90 bp, Artificial Sequence
  Primer 3G144
ccaggctgattgaaatgcccttctgtttcaggcataaagccccaaagtcataaagtacactggcagcgcggtgtaggctggagctgcttc

SEQ ID NO: 44 - DNA, 93 bp, Artificial Sequence
  Primer 3G145
gcatggctactcctcaacgacgttgtctgttagtggttgaattatttgctcaggatgtggcattgtcaagggcattccggggatccgtcgacc

SEQ ID NO: 45 - DNA, 25 bp, Artificial Sequence
  Primer YCIKUp
gataataccgcgttcatcctgggcc

SEQ ID NO: 46 - DNA, 25 bp, Artificial Sequence
  Primer YCIKDn
gcgagttcacttcatgggcgtccat

SEQ ID NO: 47 - DNA, 100 bp, Artificial Sequence
  Primer pta 1
atgtcgagtaagttagtactggttctgaactgcggtagttcttcactgaaatttgccatcatcgatgcagtaaatggtgatgtgtaggctggagctgctt

SEQ ID NO: 48 - DNA, 100 bp, Artificial Sequence
  Primer ack-pta 2
ttactgctgctgtgcagactgaatcgcagtcagcgcgatggtgtagacgatatcgtcaaccagtgcgccacgggacaggtcatatgaatatcctccttag

SEQ ID NO: 49 - DNA, 20 bp, Artificial Sequence
  Primer ack-U
attcattgagtcgtcaaatt

SEQ ID NO: 50 - DNA, 20 bp, Artificial Sequence
  Primer ack-D
attgcggacatagcgcaaat

SEQ ID NO: 51 - DNA, 98 bp, Artificial Sequence
  Primer ptsHFRT1
atgttccagcaagaagttaccattaccgctccgaacggtctgcacacccgccctgctgcccagtttgtaaaagaagctgtgtaggctggagctgcttc

SEQ ID NO: 52 - DNA, 97 bp, Artificial Sequence
  Primer crrFRT11
ttacttcttgatgcggataaccggggtttcacccacggttacgctaccggacagtttgatcagttctttgatttcgtcatatgaatatcctccttag

SEQ ID NO: 53 - DNA, 36 bp, Artificial Sequence
  Primer crrR
cctgttttgtgctcagctcatcagtggcttgctgaa

SEQ ID NO: 54 - DNA, 13669 bp, Artificial sequence
  Plasmid pSYCO101
tagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcatttaaatagtcaaaagcctccgaccggaggcttttgactgctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttgcgggagagaatgatgaacaagagccaacaagttcagacaatcaccctggccgccgcccagcaaatggcggcggcggtggaaaaaaaagccactgagatcaacgtggcggtggtgttttccgtagttgaccgcggaggcaacacgctgcttatccagcggatggacgaggccttcgtctccagctgcgatatttccctgaataaagcctggagcgcctgcagcctgaagcaaggtacccatgaaattacgtcagcggtccagccaggacaatctctgtacggtctgcagctaaccaaccaacagcgaattattatttttggcggcggcctgccagttatttttaatgagcaggtaattggcgccgtcggcgttagcggcggtacggtcgagcaggatcaattattagcccagtgcgccctggattgtttttccgcattataacctgaagcgagaaggtatattatgagctatcgtatgttccgccaggcattctgagtgttaacgaggggaccgtcatgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcaactgcgtacgctcggcgccaacgccgggcagctggttaaagtcctgccgttaagtgagagaaactgaatgtatcgtatctatacccgcaccggggataaaggcaccaccgccctgtacggcggcagccgcatcgagaaagaccatattcgcgtcgaggcctacggcaccgtcgatgaactgatatcccagctgggcgtctgctacgccacgacccgcgacgccgggctgcgggaaagcctgcaccatattcagcagacgctgttcgtgctgggggctgaactggccagcgatgcgcggggcctgacccgcctgagccagacgatcggcgaagaggagatcaccgccctggagcggcttatcgaccgcaatatggccgagagcggcccgttaaaacagttcgtgatcccggggaggaatctcgcctctgcccagctgcacgtggcgcgcacccagtcccgtcggctcgaacgcctgctgacggccatggaccgcgcgcatccgctgcgcgacgcgctcaaacgctacagcaatcgcctgtcggatgccctgttctccatggcgcgaatcgaagagactaggcctgatgcttgcgcttgaactggcctagcaaacacagaaaaaagcccgcacctgacagtgcgggctttttttttcctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttcaccttttgagccgatgaacaatgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataaggcggtattcctgtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaagcggaggtcagcatgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaacgggcgctcgcgccagcctctaggtacaaataaaaaaggcacgtcagatgacgtgccttttttcttgtctagagtactggcgaaagggggatgtgctgcaaggcgattaagttgggtaacgccagggttttcccagtcacgacgttgtaaaacgacggccagtgaattcgagctcggtacccggggcggccgcgctagcgcccgatccagctggagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgcctgcagcccgggttaccatttcaacagatcgtccttagcatataagtagtcgtcaaaaatgaattcaacttcgtctgtttcggcattgtagccgccaactctgatggattcgtggtttttgacaatgatgtcacagcctttttcctttaggaagtccaagtcgaaagtagtggcaataccaatgatcttacaaccggcggcttttccggcggcaatacctgctggagcgtcttcaaatactactaccttagatttggaagggtcttgctcattgatcggatatcctaagccattcctgcccttcagatatggttctggatgaggcttaccctgtttgacatcattagcggtaatgaagtactttggtctcctgattcccagatgctcgaaccatttttgtgccatatcacgggtaccggaagttgccacagcccatttctcttttggtagagcgttcaaagcgttgcacagcttaactgcacctgggacttcaatggatttttcaccgtacttgaccggaatttcagcttctaatttgttaacatactcttcattggcaaagtctggagcgaacttagcaatggcatcaaacgttctccaaccatgcgagacttggataacgtgttcagcatcgaaataaggtttgtccttaccgaaatccctccagaatgcagcaatggctggttgagagatgataatggtaccgtcgacgtcgaacaaagcggcgttaactttcaaagatagaggtttagtagtcaatcccataattctagtctgtttcctggatccaataaatctaatcttcatgtagatctaattcttcaatcatgtccggcaggttcttcattgggtagttgttgtaaacgatttggtatacggcttcaaataatgggaagtcttcgacagagccacatgtttccaaccattcgtgaacttctttgcaggtaattaaaccttgagcggattggccattcaacaactccttttcacattcccaggcgtccttaccagaagtagccattagcctagcaaccttgacgtttctaccaccagcgcaggtggtgatcaaatcagcaacaccagcagactcttggtagtatgtttcttctctagattctgggaaaaacatttgaccgaatctgatgatctcacccaaaccgactctttggatggcagcagaagcgttgttaccccagcctagaccttcgacgaaaccacaacctaaggcaacaacgttcttcaaagcaccacagatggagataccagcaacatcttcgatgacactaacgtggaagtaaggtctgtggaacaaggcctttagaaccttatggtcgacgtccttgccctcgcctctgaaatcctttggaatgtggtaagcaactgttgtttcagaccagtgttcttgagcgacttcggtggcaatgttagcaccagatagagcaccacattgaatacctagttcctcagtgatgtaagaggatagcaattggacacctttagcaccaacttcaaaaccctttagacaggagatagctctgacgtgtgaatcaacatgacctttcaattggctacagatacggggcaaaaattgatgtggaatgttgaaaacgatgatgtcgacatccttgactgaatcaatcaagtctggattagcaaccaaattgtcgggtagagtgatgccaggcaagtatttcacgttttgatgtctagtatttatgatttcagtcaatttttcaccattgatctcttcttcgaacacccacatttgtactattggagcgaaaacttctgggtatcccttacaattttcggcaaccaccttggcaatagtagtaccccagttaccagatccaatcacagtaaccttgaaaggcttttcggcagccttcaaagaaacagaagaggaacttctctttctaccagcattcaagtggccggaagttaagtttaatctatcagcagcagcagccatggaattgtcctccttactagtcatggtctgtttcctgtgtgaaattgttatccgctcacaattccacacattatacgagccggatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcacgctagccagtacatttaaatggtaccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagct

SEQ ID NO: 55 - DNA, 13543 bp, artificial sequence
  Plasmid pSYCO103
tagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcatttaaatagtcaaaagcctccgaccggaggcttttgactgctaggcgatctgtgctgtttgccacggtatgcagcaccagcgcgagattatgggctcgcacgctcgactgtcggacgggggcactggaacgagaagtcaggcgagccgtcacgcccttgactatgccacatcctgagcaaataattcaaccactaaacaaatcaaccgcgtttcccggaggtaaccaagcttgcgggagagaatgatgaacaagagccaacaagttcagacaatcaccctggccgccgcccagcaaatggcggcggcggtggaaaaaaaagccactgagatcaacgtggcggtggtgttttccgtagttgaccgcggaggcaacacgctgcttatccagcggatggacgaggccttcgtctccagctgcgatatttccctgaataaagcctggagcgcctgcagcctgaagcaaggtacccatgaaattacgtcagcggtccagccaggacaatctctgtacggtctgcagctaaccaaccaacagcgaattattatttttggcggcggcctgccagttatttttaatgagcaggtaattggcgccgtcggcgttagcggcggtacggtcgagcaggatcaattattagcccagtgcgccctggattgtttttccgcattataacctgaagcgagaaggtatattatgagctatcgtatgttccgccaggcattctgagtgttaacgaggggaccgtcatgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcaactgcgtacgctcggcgccaacgccgggcagctggttaaagtcctgccgttaagtgagagaaactgaatgtatcgtatctatacccgcaccggggataaaggcaccaccgccctgtacggcggcagccgcatcgagaaagaccatattcgcgtcgaggcctacggcaccgtcgatgaactgatatcccagctgggcgtctgctacgccacgacccgcgacgccgggctgcgggaaagcctgcaccatattcagcagacgctgttcgtgctgggggctgaactggccagcgatgcgcggggcctgacccgcctgagccagacgatcggcgaagaggagatcaccgccctggagcggcttatcgaccgcaatatggccgagagcggcccgttaaaacagttcgtgatcccggggaggaatctcgcctctgcccagctgcacgtggcgcgcacccagtcccgtcggctcgaacgcctgctgacggccatggaccgcgcgcatccgctgcgcgacgcgctcaaacgctacagcaatcgcctgtcggatgccctgttctccatggcgcgaatcgaagagactaggcctgatgcttgcgcttgaactggcctagcaaacacagaaaaaagcccgcacctgacagtgcgggctttttttttcctaggcgatctgtgctgtttgccacggtatgcagcaccagcgcgagattatgggctcgcacgctcgactgtcggacgggggcactggaacgagaagtcaggcgagccgtcacgcccttgactatgccacatcctgagcaaataattcaaccactaaacaaatcaaccgcgtttcccggaggtaaccaagcttcaccttttgagccgatgaacaatgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataaggcggtattcctgtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaagcggaggtcagcatgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaacgggcgctcgcgccagcctctaggtacaaataaaaaaggcacgtcagatgacgtgccttttttcttgtctagcgtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgactagtaaggaggacaattccatggctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattagatttattggatccaggaaacagactagaattatgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaacccgggctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactccagctggatcgggcgctagagtatacatttaaatggtaccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagct

SEQ ID NO: 56 - DNA, 13543 bp, Artificial sequence
  Plasmid pSYCO106
tagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcatttaaatagtcaaaagcctccgaccggaggcttttgactgctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttgcgggagagaatgatgaacaagagccaacaagttcagacaatcaccctggccgccgcccagcaaatggcggcggcggtggaaaaaaaagccactgagatcaacgtggcggtggtgttttccgtagttgaccgcggaggcaacacgctgcttatccagcggatggacgaggccttcgtctccagctgcgatatttccctgaataaagcctggagcgcctgcagcctgaagcaaggtacccatgaaattacgtcagcggtccagccaggacaatctctgtacggtctgcagctaaccaaccaacagcgaattattatttttggcggcggcctgccagttatttttaatgagcaggtaattggcgccgtcggcgttagcggcggtacggtcgagcaggatcaattattagcccagtgcgccctggattgtttttccgcattataacctgaagcgagaaggtatattatgagctatcgtatgttccgccaggcattctgagtgttaacgaggggaccgtcatgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcaactgcgtacgctcggcgccaacgccgggcagctggttaaagtcctgccgttaagtgagagaaactgaatgtatcgtatctatacccgcaccggggataaaggcaccaccgccctgtacggcggcagccgcatcgagaaagaccatattcgcgtcgaggcctacggcaccgtcgatgaactgatatcccagctgggcgtctgctacgccacgacccgcgacgccgggctgcgggaaagcctgcaccatattcagcagacgctgttcgtgctgggggctgaactggccagcgatgcgcggggcctgacccgcctgagccagacgatcggcgaagaggagatcaccgccctggagcggcttatcgaccgcaatatggccgagagcggcccgttaaaacagttcgtgatcccggggaggaatctcgcctctgcccagctgcacgtggcgcgcacccagtcccgtcggctcgaacgcctgctgacggccatggaccgcgcgcatccgctgcgcgacgcgctcaaacgctacagcaatcgcctgtcggatgccctgttctccatggcgcgaatcgaagagactaggcctgatgcttgcgcttgaactggcctagcaaacacagaaaaaagcccgcacctgacagtgcgggctttttttttcctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttcaccttttgagccgatgaacaatgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataaggcggtattcctgtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaagcggaggtcagcatgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaacgggcgctcgcgccagcctctaggtacaaataaaaaaggcacgtcagatgacgtgccttttttcttgtctagcgtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgactagtaaggaggacaattccatggctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattagatttattggatccaggaaacagactagaattatgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaacccgggctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactccagctggatcgggcgctagagtatacatttaaatggtaccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagct

SEQ ID NO: 57 - DNA, 13402 bp, Artificial sequence
  Plasmid pSYCO109
tagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcatttaaatagtcaaaagcctccgaccggaggcttttgactgctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttgcgggagagaatgatgaacaagagccaacaagttcagacaatcaccctggccgccgcccagcaaatggcggcggcggtggaaaaaaaagccactgagatcaacgtggcggtggtgttttccgtagttgaccgcggaggcaacacgctgcttatccagcggatggacgaggccttcgtctccagctgcgatatttccctgaataaagcctggagcgcctgcagcctgaagcaaggtacccatgaaattacgtcagcggtccagccaggacaatctctgtacggtctgcagctaaccaaccaacagcgaattattatttttggcggcggcctgccagttatttttaatgagcaggtaattggcgccgtcggcgttagcggcggtacggtcgagcaggatcaattattagcccagtgcgccctggattgtttttccgcattataacctgaagcgagaaggtatattatgagctatcgtatgttccgccaggcattctgagtgttaacgaggggaccgtcatgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcaactgcgtacgctcggcgccaacgccgggcagctggttaaagtcctgccgttaagtgagagaaactgaatgtatcgtatctatacccgcaccggggataaaggcaccaccgccctgtacggcggcagccgcatcgagaaagaccatattcgcgtcgaggcctacggcaccgtcgatgaactgatatcccagctgggcgtctgctacgccacgacccgcgacgccgggctgcgggaaagcctgcaccatattcagcagacgctgttcgtgctgggggctgaactggccagcgatgcgcggggcctgacccgcctgagccagacgatcggcgaagaggagatcaccgccctggagcggcttatcgaccgcaatatggccgagagcggcccgttaaaacagttcgtgatcccggggaggaatctcgcctctgcccagctgcaccctgatgcttgcgcttgaactggcctagcaaacacagaaaaaagcccgcacctgacagtgcgggctttttttttcctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttcaccttttgagccgatgaacaatgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataaggcggtattcctgtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaagcggaggtcagcatgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaacgggcgctcgcgccagcctctaggtacaaataaaaaaggcacgtcagatgacgtgccttttttcttgtctagcgtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgactagtaaggaggacaattccatggctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattagatttattggatccaggaaacagactagaattatgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaacccgggctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactccagctggatcgggcgctagagtatacatttaaatggtaccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagct

SEQ ID NO: 58 - DNA, 1176 bp, Saccharomyces cerevisiae
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 59 - protein, 391 aa, Saccharomyces cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 60 - DNA, 1323 bp, Saccharomyces cerevisiae
atgcttgctgtcagaagattaacaagatacacattccttaagcgaacgcatccggtgttatatactcgtcgtgcatataaaattttgccttcaagatctactttcctaagaagatcattattacaaacacaactgcactcaaagatgactgctcatactaatatcaaacagcacaaacactgtcatgaggaccatcctatcagaagatcggactctgccgtgtcaattgtacatttgaaacgtgcgcccttcaaggttacagtgattggttctggtaactgggggaccaccatcgccaaagtcattgcggaaaacacagaattgcattcccatatcttcgagccagaggtgagaatgtgggtttttgatgaaaagatcggcgacgaaaatctgacggatatcataaatacaagacaccagaacgttaaatatctacccaatattgacctgccccataatctagtggccgatcctgatcttttacactccatcaagggtgctgacatccttgttttcaacatccctcatcaatttttaccaaacatagtcaaacaattgcaaggccacgtggcccctcatgtaagggccatctcgtgtctaaaagggttcgagttgggctccaagggtgtgcaattgctatcctcctatgttactgatgagttaggaatccaatgtggcgcactatctggtgcaaacttggcaccggaagtggccaaggagcattggtccgaaaccaccgtggcttaccaactaccaaaggattatcaaggtgatggcaaggatgtagatcataagattttgaaattgctgttccacagaccttacttccacgtcaatgtcatcgatgatgttgctggtatatccattgccggtgccttgaagaacgtcgtggcacttgcatgtggtttcgtagaaggtatgggatggggtaacaatgcctccgcagccattcaaaggctgggtttaggtgaaattatcaagttcggtagaatgtttttcccagaatccaaagtcgagacctactatcaagaatccgctggtgttgcagatctgatcaccacctgctcaggcggtagaaacgtcaaggttgccacatacatggccaagaccggtaagtcagccttggaagcagaaaaggaattgcttaacggtcaatccgcccaagggataatcacatgcagagaagttcacgagtggctacaaacatgtgagttgacccaagaattcccattattcgaggcagtctaccagatagtctacaacaacgtccgcatggaagacctaccggagatgattgaagagctagacatcgatgacgaatag

SEQ ID NO: 61 - protein, 440 aa, Saccharomyces cerevisiae
MLAVRRLTRYTFLKRTHPVLYTRRAYKILPSRSTFLRRSLLQTQLHSKMTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPLFEAVYQIVYNNVRMEDLPEMIEELDIDDE

SEQ ID NO: 62 - DNA, 816 bp, Saccharomyces cerevisiae
atgaaacgtttcaatgttttaaaatatatcagaacaacaaaagcaaatatacaaaccatcgcaatgcctttgaccacaaaacctttatctttgaaaatcaacgccgctctattcgatgttgacggtaccatcatcatctctcaaccagccattgctgctttctggagagatttcggtaaagacaagccttacttcgatgccgaacacgttattcacatctctcacggttggagaacttacgatgccattgccaagttcgctccagactttgctgatgaagaatacgttaacaagctagaaggtgaaatcccagaaaagtacggtgaacactccatcgaagttccaggtgctgtcaagttgtgtaatgctttgaacgccttgccaaaggaaaaatgggctgtcgccacctctggtacccgtgacatggccaagaaatggttcgacattttgaagatcaagagaccagaatacttcatcaccgccaatgatgtcaagcaaggtaagcctcacccagaaccatacttaaagggtagaaacggtttgggtttcccaattaatgaacaagacccatccaaatctaaggttgttgtctttgaagacgcaccagctggtattgctgctggtaaggctgctggctgtaaaatcgttggtattgctaccactttcgatttggacttcttgaaggaaaagggttgtgacatcattgtcaagaaccacgaatctatcagagtcggtgaatacaacgctgaaaccgatgaagtcgaattgatctttgatgactacttatacgctaaggatgacttgttgaaatggtaa

SEQ ID NO: 63 - protein, 271 aa, Saccharomyces cerevisiae
MKRFNVLKYIRTTKANIQTIAMPLTTKPLSLKINAALFDVDGTIIISQPAIAAFWRDFGKDKPYFDAEHVIHISHGWRTYDAIAKFAPDFADEEYVNKLEGEIPEKYGEHSIEVPGAVKLCNALNALPKEKWAVATSGTRDMAKKWFDILKIKRPEYFITANDVKQGKPHPEPYLKGRNGLGFPINEQDPSKSKVVVFEDAPAGIAAGKAAGCKIVGIATTFDLDFLKEKGCDIIVKNHESIRVGEYNAETDEVELIFDDYLYAKDDLLKW

SEQ ID NO: 64 - DNA, 753 bp, Saccharomyces cerevisiae
atgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaa

SEQ ID NO: 65 - protein, 250 aa, Saccharomyces cerevisiae
MGLTTKPLSLKVNAALFDVDGTIIISQPAIAAFWRDFGKDKPYFDAEHVIQVSHGWRTFDAIAKFAPDFANEEYVNKLEAEIPVKYGEKSIEVPGAVKLCNALNALPKEKWAVATSGTRDMAQKWFEHLGIRRPKYFITANDVKQGKPHPEPYLKGRNGLGYPINEQDPSKSKVVVFEDAPAGIAAGKAAGCKIIGIATTFDLDFLKEKGCDIIVKNHESIRVGGYNAETDEVEFIFDDYLYAKDDLLKW

SEQ ID NO: 66 - DNA, 1668 bp, Klebsiella pneumoniae
atgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataa

SEQ ID NO: 67 - DNA, 585 bp, Klebsiella pneumoniae
gtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtga

SEQ ID NO: 68 - DNA, 426 bp, Klebsiella pneumoniae
atgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaa

SEQ ID NO: 69 - DNA, 1824 bp, Klebsiella pneumoniae
atgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaa

SEQ ID NO: 70 - DNA, 1440 bp, Escherichia coli
  CDS (1)..(1440)
atgtcagtacccgttcaacatcctatgtatatcgatggacagtttgttacctggcgtggagacgcatggattgatgtggtaaaccctgctacagaggctgtcatttcccgcatacccgatggtcaggccgaggatgcccgtaaggcaatcgatgcagcagaacgtgcacaaccagaatgggaagcgttgcctgctattgaacgcgccagttggttgcgcaaaatctccgccgggatccgcgaacgcgccagtgaaatcagtgcgctgattgttgaagaagggggcaagatccagcagctggctgaagtcgaagtggcttttactgccgactatatcgattacatggcggagtgggcacggcgttacgagggcgagattattcaaagcgatcgtccaggagaaaatattcttttgtttaaacgtgcgcttggtgtgactaccggcattctgccgtggaacttcccgttcttcctcattgcccgcaaaatggctcccgctcttttgaccggtaataccatcgtcattaaacctagtgaatttacgccaaacaatgcgattgcattcgccaaaatcgtcgatgaaataggccttccgcgcggcgtgtttaaccttgtactggggcgtggtgaaaccgttgggcaagaactggcgggtaacccaaaggtcgcaatggtcagtatgacaggcagcgtctctgcaggtgagaagatcatggcgactgcggcgaaaaacatcaccaaagtgtgtctggaattggggggtaaagcaccagctatcgtaatggacgatgccgatcttgaactggcagtcaaagccatcgttgattcacgcgtcattaatagtgggcaagtgtgtaactgtgcagaacgtgtttatgtacagaaaggcatttatgatcagttcgtcaatcggctgggtgaagcgatgcaggcggttcaatttggtaaccccgctgaacgcaacgacattgcgatggggccgttgattaacgccgcggcgctggaaagggtcgagcaaaaagtggcgcgcgcagtagaagaaggggcgagagtggcgttcggtggcaaagcggtagaggggaaaggatattattatccgccgacattgctgctggatgttcgccaggaaatgtcgattatgcatgaggaaacctttggcccggtgctgccagttgtcgcatttgacacgctggaagatgctatctcaatggctaatgacagtgattacggcctgacctcatcaatctatacccaaaatctgaacgtcgcgatgaaagccattaaagggctgaagtttggtgaaacttacatcaaccgtgaaaacttcgaagctatgcaaggcttccacgccggatggcgtaaatccggtattggcggcgcagatggtaaacatggcttgcatgaatatctgcagacccaggtggtttatttacagtcttaa

SEQ ID NO: 71 - protein, 479 aa, Escherichia coli
MSVPVQHPMYIDGQFVTWRGDAWIDVVNPATEAVISRIPDGQAEDARKAIDAAERAQPEWEALPAIERASWLRKISAGIRERASEISALIVEEGGKIQQLAEVEVAFTADYIDYMAEWARRYEGEIIQSDRPGENILLFKRALGVTTGILPWNFPFFLIARKMAPALLTGNTIVIKPSEFTPNNAIAFAKIVDEIGLPRGVFNLVLGRGETVGQELAGNPKVAMVSMTGSVSAGEKIMATAAKNITKVCLELGGKAPAIVMDDADLELAVKAIVDSRVINSGQVCNCAERVYVQKGIYDQFVNRLGEAMQAVQFGNPAERNDIAMGPLINAAALERVEQKVARAVEEGARVAFGGKAVEGKGYYYPPTLLLDVRQEMSIMHEETFGPVLPVVAFDTLEDAISMANDSDYGLTSSIYTQNLNVAMKAIKGLKFGETYINRENFEAMQGFHAGWRKSGIGGADGKHGLHEYLQTQVVYLQS

SEQ ID NO: 72 - DNA, 1539 bp, Escherichia coli
  CDS (1)..(1539)
atgaccaataatcccccttcagcacagattaagcccggcgagtatggtttccccctcaagttaaaagcccgctatgacaactttattggcggcgaatgggtagcccctgccgacggcgagtattaccagaatctgacgccggtgaccgggcagctgctgtgcgaagtggcgtcttcgggcaaacgagacatcgatctggcgctggatgctgcgcacaaagtgaaagataaatgggcgcacacctcggtgcaggatcgtgcggcgattctgtttaagattgccgatcgaatggaacaaaacctcgagctgttagcgacagctgaaacctgggataacggcaaacccattcgcgaaaccagtgctgcggatgtaccgctggcgattgaccatttccgctatttcgcctcgtgtattcgggcgcaggaaggtgggatcagtgaagttgatagcgaaaccgtggcctatcatttccatgaaccgttaggcgtggtggggcagattatcccgtggaacttcccgctgctgatggcgagctggaaaatggctcccgcgctggcggcgggcaactgtgtggtgctgaaacccgcacgtcttaccccgctttctgtactgctgctaatggaaattgtcggtgatttactgccgccgggcgtggtgaacgtggtcaatggcgcaggtggggtaattggcgaatatctggcgacctcgaaacgcatcgccaaagtggcgtttaccggctcaacggaagtgggccaacaaattatgcaatacgcaacgcaaaacattattccggtgacgctggagttgggcggtaagtcgccaaatatcttctttgctgatgtgatggatgaagaagatgcctttttcgataaagcgctggaaggctttgcactgtttgcctttaaccagggcgaagtttgcacctgtccgagtcgtgctttagtgcaggaatctatctacgaacgctttatggaacgcgccatccgccgtgtcgaaagcattcgtagcggtaacccgctcgacagcgtgacgcaaatgggcgcgcaggtttctcacgggcaactggaaaccatcctcaactacattgatatcggtaaaaaagagggcgctgacgtgctcacaggcgggcggcgcaagctgctggaaggtgaactgaaagacggctactacctcgaaccgacgattctgtttggtcagaacaatatgcgggtgttccaggaggagatttttggcccggtgctggcggtgaccaccttcaaaacgatggaagaagcgctggagctggcgaacgatacgcaatatggcctgggcgcgggcgtctggagccgcaacggtaatctggcctataagatggggcgcggcatacaggctgggcgcgtgtggaccaactgttatcacgcttacccggcacatgcggcgtttggtggctacaaacaatcaggtatcggtcgcgaaacccacaagatgatgctggagcattaccagcaaaccaagtgcctgctggtgagctactcggataaaccgttggggctgttctga

SEQ ID NO: 73 - protein, 512 aa, Escherichia coli
MTNNPPSAQIKPGEYGFPLKLKARYDNFIGGEWVAPADGEYYQNLTPVTGQLLCEVASSGKRDIDLALDAAHKVKDKWAHTSVQDRAAILFKIADRMEQNLELLATAETWDNGKPIRETSAADVPLAIDHFRYFASCIRAQEGGISEVDSETVAYHFHEPLGVVGQIIPWNFPLLMASWKMAPALAAGNCVVLKPARLTPLSVLLLMEIVGDLLPPGVVNVVNGAGGVIGEYLATSKRIAKVAFTGSTEVGQQIMQYATQNIIPVTLELGGKSPNIFFADVMDEEDAFFDKALEGFALFAFNQGEVCTCPSRALVQESIYERFMERAIRRVESIRSGNPLDSVTQMGAQVSHGQLETILNYIDIGKKEGADVLTGGRRKLLEGELKDGYYLEPTILFGQNNMRVFQEEIFGPVLAVTTFKTMEEALELANDTQYGLGAGVWSRNGNLAYKMGRGIQAGRVWTNCYHAYPAHAAFGGYKQSGIGRETHKMMLEHYQQTKCLLVSYSDKPLGLF

SEQ ID NO: 74 - DNA, 1488 bp, Escherichia coli
  CDS (1)..(1488)
atgaattttcatcatctggcttactggcaggataaagcgttaagtctcgccattgaaaaccgcttatttattaacggtgaatatactgctgcggcggaaaatgaaacctttgaaaccgttgatccggtcacccaggcaccgctggcgaaaattgcccgcggcaagagcgtcgatatcgaccgtgcgatgagcgcagcacgcggcgtatttgaacgcggcgactggtcactctcttctccggctaaacgtaaagcggtactgaataaactcgccgatttaatggaagcccacgccgaagagctggcactgctggaaactctcgacaccggcaaaccgattcgtcacagtctgcgtgatgatattcccggcgcggcgcgcgccattcgctggtacgccgaagcgatcgacaaagtgtatggcgaagtggcgaccaccagtagccatgagctggcgatgatcgtgcgtgaaccggtcggcgtgattgccgccatcgtgccgtggaacttcccgctgttgctgacttgctggaaactcggcccggcgctggcggcgggaaacagcgtgattctaaaaccgtctgaaaaatcaccgctcagtgcgattcgtctcgcggggctggcgaaagaagcaggcttgccggatggtgtgttgaacgtggtgacgggttttggtcatgaagccgggcaggcgctgtcgcgtcataacgatatcgacgccattgcctttaccggttcaacccgtaccgggaaacagctgctgaaagatgcgggcgacagcaacatgaaacgcgtctggctggaagcgggcggcaaaagcgccaacatcgttttcgctgactgcccggatttgcaacaggcggcaagcgccaccgcagcaggcattttctacaaccagggacaggtgtgcatcgccggaacgcgcctgttgctggaagagagcatcgccgatgaattcttagccctgttaaaacagcaggcgcaaaactggcagccgggccatccacttgatcccgcaaccaccatgggcaccttaatcgactgcgcccacgccgactcggtccatagctttattcgggaaggcgaaagcaaagggcaactgttgttggatggccgtaacgccgggctggctgccgccatcggcccgaccatctttgtggatgtggacccgaatgcgtccttaagtcgcgaagagattttcggtccggtgctggtggtcacgcgtttcacatcagaagaacaggcgctacagcttgccaacgacagccagtacggccttggcgcggcggtatggacgcgcgacctctcccgcgcgcaccgcatgagccgacgcctgaaagccggttccgtcttcgtcaataactacaacgacggcgatatgaccgtgccgtttggcggctataagcagagcggcaacggtcgcgacaaatccctgcatgcccttgaaaaattcactgaactgaaaaccatctggataagcctggaggcctga

SEQ ID NO: 75 - protein, 495 aa, Escherichia coli
MNFHHLAYWQDKALSLAIENRLFINGEYTAAAENETFETVDPVTQAPLAKIARGKSVDIDRAMSAARGVFERGDWSLSSPAKRKAVLNKLADLMEAHAEELALLETLDTGKPIRHSLRDDIPGAARAIRWYAEAIDKVYGEVATTSSHELAMIVREPVGVIAAIVPWNFPLLLTCWKLGPALAAGNSVILKPSEKSPLSAIRLAGLAKEAGLPDGVLNVVTGFGHEAGQALSRHNDIDAIAFTGSTRTGKQLLKDAGDSNMKRVWLEAGGKSANIVFADCPDLQQAASATAAGIFYNQGQVCIAGTRLLLEESIADEFLALLKQQAQNWQPGHPLDPATTMGTLIDCAHADSVHSFIREGESKGQLLLDGRNAGLAAAIGPTIFVDVDPNASLSREEIFGPVLVVTRFTSEEQALQLANDSQYGLGAAVWTRDLSRAHRMSRRLKAGSVFVNNYNDGDMTVPFGGYKQSGNGRDKSLHALEKFTELKTIWISLEA

SEQ ID NO: 76 - DNA, 1164 bp, Escherichia coli
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 77 - DNA, 35 bp, Artificial Sequence
  Primer Afor
gcgcgcaagcttatgtcagtacccgttcaacatcc

SEQ ID NO: 78 - DNA, 38 bp, Artificial Sequence
  Primer Arev
gcgcgcaagcttttaagactgtaaataaaccacctggg

SEQ ID NO: 79 - DNA, 35 bp, Artificial Sequence
  Primer Bfor
gcgcgcaagcttatgaccaataatcccccttcagc

SEQ ID NO: 80 - DNA, 30 bp, Artificial Sequence
  Primer Brev
gcgcgcaagctttcagaacagccccaacgg

SEQ ID NO: 81 - DNA, 39 bp, Artificial Sequence
  Primer Hfor
gcgcgcaagcttatgaattttcatcatctggcttactgg

SEQ ID NO: 82 - DNA, 32 bp, Artificial Sequence
  Primer Hrev
gcgcgcaagctttcaggcctccaggcttatcc

(19) United States
(12) Patent Application Publication
(10) Pub. No.: US 2011/0144377 A1
(43) Pub. Date: Jun. 16, 2011

(54) PROCESS FOR THE BIOLOGICAL PRODUCTION OF 3-HYDROXYPROPIONIC ACID WITH HIGH YIELD

(75) Inventors: Andrew C. Eliot, Wilmington, DE (US); Tina K. Van Dyk, Wilmington, DE (US)

(73) Assignee: E. I. DU PONT NEMOURS AND COMPANY, Wilmington, DE (US)

(21) Appl. No.: 12/815,461

(22) Filed: Jun. 15, 2010

Related U.S. Application Data
(60) Provisional application No. 61/187,476, filed on Jun. 16, 2009.

(51) Int. Cl. C12P 7/42; C12N 1/20; C07C 59/01; C07C 59/40; C07C 55/08; C07C 69/52; C07C 69/34

(52) U.S. Cl. 560/190; 435/252.33; 435/146; 562/579; 562/598; 562/590; 560/205

(57) ABSTRACT

The present invention provides a microorganism useful for biologically producing 3-hydroxypropionic acid from a fermentable carbon source. Further, the microorganism comprises disruptions in specified genes and alterations in the expression levels of specified genes that are useful in a higher yielding process to produce 3-hydroxypropionic acid, compositions comprising renewably sourced 3-hydroxypropionic acid provided by said microorganism, and industrial relevant products made using such renewably sourced 3-hydroxypropionic acid.

### FIELD OF THE INVENTION

The invention relates to the fields of microbiology and fermentation. More specifically, a process for the bioconversion of a fermentable carbon source to 3-hydroxypropionic acid by a single microorganism is provided.

### BACKGROUND OF THE INVENTION

Organic chemicals such as organic acids, esters, and polyols can be used to synthesize plastic materials and other products. To meet the increasing demand for organic chemicals, more efficient, cost effective and environmentally sound production methods are being developed which utilize raw materials based on carbohydrates rather than hydrocarbons. For example, certain bacteria have been used to produce large quantities of 1,3-propanediol (U.S. Pat. No. 7,371,558).

3-hydroxypropionic acid (3-HP) is an organic acid. Although several chemical synthesis routes have been described to produce 3-HP, few biological systems have been developed that provide more efficient, cost effective and environmentally sound production mechanisms (WO 01/16346 to Suthers, et al.; U.S. Pat. No. 7,393,676 B2). 3-HP has utility for specialty synthesis and can be converted to commercially important intermediates by known art in the chemical industry, e.g., acrylic acid by dehydration, malonic acid by oxidation, esters by esterification reactions with alcohols, and reduction to 1,3-propanediol.

Thus, there remains a need to produce 3-HP in high yield by more efficient, cost effective and environmentally sound production methods in which raw materials are utilized that are based on carbohydrates rather than hydrocarbons. Such produced 3-HP can then be coverted to other commercially relevant intermediates.

### SUMMARY OF THE INVENTION

Applicants have solved the stated problem. The present invention provides for bioconverting a fermentable carbon source to 3-HP with the use of a single microorganism. The yield obtained is, 2×, 5×, 10×, 20×, 50×, 100×, or 200× that of the control strain. Glucose is used as a model substrate and Escherichia coli is used as the model host microorganism with the useful genetic modifications and disruptions detailed herein.

### BRIEF DESCRIPTION OF THE FIGURES AND SEQUENCE DESCRIPTIONS

The invention can be more fully understood from the following detailed description, the Figures, and the accompanying sequence descriptions that form a part of this application.

FIG. 1 is a diagram of a pathway for making 3-HP.

The following sequences conform with 37 C.F.R. 1.821 1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a bis), and Section 208 and Annex C of the Administrative Instructions). The symbols and format used for nucleotide and amino acid sequence data comply with the rules set forth in 37 C.F.R. §1.822.

SEQ ID NO:1 is the partial nucleotide sequence of pLoxCat27 encoding the loxP511-Cat-loxP511 cassette.

SEQ ID NO:2-3 are oligonucleotide primers used to construct the arcA disruption.

SEQ ID NOs:4-5 are oligonucleotide primers used to confirm disruption of arcA.

SEQ ID NO:6 is the partial nucleotide sequence of pLoxCat1 encoding the loxP-Cat-loxP cassette.

SEQ ID NOs:7-8 are oligonucleotide primers used to construct pR6KgalP, the template plasmid for trc promoter replacement of the chromosomal galP promoter.

SEQ ID NOs:9-10 are oligonucleotide primers used to construct pR6Kglk, the template plasmid for trc promoter replacement of the chromosomal glk promoter.

SEQ ID NO:11 is the nucleotide sequence of the loxP-Cat-/oxP—Trc cassette.

SEQ ID NOs:12-13 are oligonucleotide primers used to confirm integration of SEQ ID NO:11 for replacement of the chromosomal galP promoter.

SEQ ID NOs:14-15 are oligonucleotide primers used to confirm integration of SEQ ID NO:11 for replacement of the chromosomal glk promoter.

SEQ ID NOs:16-17 are oligonucleotide primers used to construct the edd disruption.

SEQ ID NOs:18-19 are oligonucleotide primers used to confirm disruption of edd.

SEQ ID NOs:20 is the nucleotide sequence for the selected trc promoter controlling glk expression.

SEQ ID NOs:21 is the partial nucleotide sequence for the standard trc promoter.

SEQ ID NOs:22-23 are the oligonucleotide primers used for amplification of gapA.

SEQ ID NOs:24-25 are the oligonucleotide primers used to alter the start codon of gapA to GTG.

SEQ ID NOs:26-27 are the oligonucleotide primers used to alter the start codon of gapA to TTG.

SEQ ID NO:28 is the nucleotide sequence for the short 1.5 GI promoter.

SEQ ID NOs:29-30 are oligonucleotide primers used for replacement of the chromosomal gapA promoter with the short 1.5 GI promoter.

SEQ ID NO:31 is the nucleotide sequence for the short 1.20 GI promoter.

SEQ ID NO:32 is the nucleotide sequence for the short 1.6 GI promoter.

SEQ ID NOs:33-34 are oligonucleotide primers used for replacement of the chromosomal gapA promoter with the short 1.20 GI promoter.

SEQ ID NO:35 is the oligonucleotide primer with SEQ ID NO 33 that is used for replacement of the chromosomal gapA promoter with the short 1.6 GI promoter.

SEQ ID NOs:36-37 are oligonucleotide primers used to construct the mgsA disruption.

SEQ ID NOs:38-39 are oligonucleotide primers used to confirm disruption of mgsA.

SEQ ID NOs:40-41 are oligonucleotide primers used for replacement of the chromosomal ppc promoter with the short 1.6 GI promoter.

SEQ ID NO:42 is an oligonucleotide primer used to confirm replacement of the ppc promoter.

SEQ ID NOs:43-44 are oligonucleotide primers used for replacement of the chromosomal yciK-btuR promoter with the short 1.6 GI promoter.

SEQ ID NOs:45-46 are oligonucleotide primers used to confirm replacement of the yciK-btuR promoter.

SEQ ID NOs:47-48 are oligonucleotide primers used to construct the pta-ackA disruption.

SEQ ID NOs:49-50 are oligonucleotide primers used to confirm disruption of pta-ackA.

SEQ ID NOs:51-52 are oligonucleotide primers used to construct the ptsHlcrr disruption.

SEQ ID NO:53 is an oligonucleotide primer used to confirm disruption of ptsHlcrr.

SEQ ID NO:54 is the nucleotide sequence for the pSYCO101 plasmid.

SEQ ID NO:55 is the nucleotide sequence for the pSYCO103 plasmid.

SEQ ID NO:56 is the nucleotide sequence for the pSYCO106 plasmid.

SEQ ID NO:57 is the nucleotide sequence for the pSYCO109 plasmid.

SEQ ID NO:58 is the nucleotide sequence of the GPD1 gene from Saccharomyces cerevisiae.

SEQ ID NO:59 is the amino acid sequence of the glycerol-3-phosphate dehydrogenase encoded by GPD1.

SEQ ID NO:60 is the nucleotide sequence of the GPD2 gene from Saccharomyces cerevisiae.

SEQ ID NO:61 is the amino acid sequence of the glycerol-3-phosphate dehydrogenase encoded by GPD2.

SEQ ID NO:62 is the nucleotide sequence of the GPP1 gene from Saccharomyces cerevisiae.

SEQ ID NO:63 is the amino acid sequence of the glycerol 3-phosphatase encoded by GPP1.

SEQ ID NO:64 is the nucleotide sequence of the GPP2 gene from Saccharomyces cerevisiae.

SEQ ID NO:65 is the amino acid sequence of the glycerol 3-phosphatase encoded by GPP2.

SEQ ID NO:66 is the nucleotide sequence of the dhaB1 gene from Klebsiella pneumoniae, which encodes the a subunit of a glycerol dehydratase.

SEQ ID NO:67 is the nucleotide sequence of the dhaB2 gene from Klebsiella pneumoniae, which encodes the β subunit of a glycerol dehydratase.

SEQ ID NO:68 is the nucleotide sequence of the dhaB3 gene from Klebsiella pneumoniae, which encodes the γ subunit of a glycerol dehydratase.

SEQ ID NO:69 is the nucleotide sequence of the dhaX gene from Klebsiella pneumoniae.

SEQ ID NO:70 is the nucleotide sequence of the aldA gene from E. coli.

SEQ ID NO:71 is the amino acid sequence of the aldehyde dehydrogenase encoded by aldA.

SEQ ID NO:72 is the nucleotide sequence of the aldB gene from E. coli.

SEQ ID NO:73 is the amino acid sequence of the aldehyde dehydrogenase encoded by aldB.

SEQ ID NO:74 is the nucleotide sequence of the aldH gene from E. coli.

SEQ ID NO:75 is the amino acid sequence of the aldehyde dehydrogenase encoded by aldH.

SEQ ID NO:76 is the nucleotide sequence of the yqhD gene from E. coli.

SEQ ID NOs:77-82 are the nucleotide sequences of primers used to amplify aldehyde dehydrogenases from E. coli as described in Example 1 herein.

### DETAILED DESCRIPTION

The following abbreviations and definitions will be used for the interpretation of the specification and the claims.

The terms “glycerol-3-phosphate dehydrogenase” and “G3PDH” refer to a polypeptide responsible for an enzyme activity that catalyzes the conversion of dihydroxyacetone phosphate (DHAP) to glycerol 3-phosphate (G3P). In vivo G3PDH may be NAD- or NADP-dependent. When specifically referring to a cofactor specific glycerol-3-phosphate dehydrogenase, the terms “NAD-dependent glycerol-3-phosphate dehydrogenase” and “NADP-dependent glycerol-3-phosphate dehydrogenase” will be used. As it is generally the case that NAD-dependent and NADP-dependent glycerol-3-phosphate dehydrogenases are able to use NAD and NADP interchangeably (for example by the gene encoded by gpsA), the terms NAD-dependent and NADP-dependent glycerol-3-phosphate dehydrogenase will be used interchangeably. The NAD-dependent enzyme (EC 1.1.1.8) is encoded, for example, by several genes including GPD1, also referred to herein as Dar1, [SEQ ID NO:58 (nucleotide); SEQ ID NO:59 (protein)], or GPD2 [SEQ ID NO:60 (nucleotide); SEQ ID NO:61 (protein)], or GPD3. The NADP-dependent enzyme (EC 1.1.1.94) is encoded by gpsA.

The terms “glycerol 3-phosphatase”, “sn-glycerol 3-phosphatase”, or “D,L-glycerol phosphatase”, and “G3P phosphatase” refer to a polypeptide responsible for an enzyme activity that catalyzes the conversion of glycerol 3-phosphate and water to glycerol and inorganic phosphate. G3P phosphatase is encoded, for example, by GPP1 [SEQ ID NO:62 (nucleotide); SEQ ID NO:63 (protein)], or GPP2 [SEQ ID NO:64 (nucleotide); SEQ ID NO:65 (protein)] (see WO 9928480 and references therein, which are herein incorporated by reference).

The term “glycerol dehydratase” or “dehydratase enzyme” will refer to any enzyme activity that catalyzes the conversion of a glycerol molecule to the product 3-hydroxypropionaldehyde. For the purposes of the present invention the dehydratase enzymes include a glycerol dehydratase (E.C. 4.2.1.30) and a diol dehydratase (E.C. 4.2.1.28) having preferred substrates of glycerol and 1,2-propanediol, respectively. Genes for dehydratase enzymes have been identified in Klebsiella pneumoniae, Citrobacter freundii, Clostridium pasteurianum, Salmonella typhimurium, and Klebsiella oxytoca. In each case, the dehydratase is composed of three subunits: the large or “α” subunit, the medium or “β” subunit, and the small or “γ” subunit. Due to the wide variation in gene nomenclature used in the literature, a comparative chart is given in Table 1 to facilitate identification. The genes are also described in, for example, Daniel et al. (FEMS Microbiol. Rev. 22, 553 (1999)) and Toraya and Mori (J. Biol. Chem. 274, 3372 (1999)). Referring to Table 1, genes encoding the large or “α” (alpha) subunit of glycerol dehydratase include dhaB1 (SEQ ID NO:66), gldA and dhaB; genes encoding the medium or “β” (beta) subunit include dhaB2 (SEQ ID NO:67), gldB, and dhaC; genes encoding the small or “γ” (gamma) subunit include dhaB3 (SEQ ID NO:68), gldC, and dhaE. Also referring to Table 1, genes encoding the large or “α” subunit of diol dehydratase include pduC and pddA; genes encoding the medium or “β” subunit include pduD and pddB; genes encoding the small or “γ” subunit include pduE and pddC

TABLE 1
Comparative chart of gene names and GenBank references for dehydratase and dehydratase linked functions.

| | GENE FUNCTION: | | | | | | | |
|---|---|---|---|---|---|---|---|---|
| ORGANISM (GenBank | regulatory | | unknown | | reactivation | | unknown | |
| Reference) | gene | base pairs | gene | base pairs | Gene | base pairs | gene | base pairs |
| K. pneumoniae (SEQ ID NO: !) | dhaR | 2209-4134 | orfW | 4112-4642 | OrfX | 4643-4996 | orfY | 6202-6630 |
| K. pneumoniae (U30903) | | | orf2c | 7116-7646 | orf2b | 6762-7115 | orf2a | 5125-5556 |
| K. pneumoniae (U60992) | | | | | GdrB | | | |
| C. freundii (U09771) | dhaR | 3746-5671 | orfW | 5649-6179 | OrfX | 6180-6533 | orfY | 7736-8164 |
| C. pasteurianum (AF051373) | | | | | | | | |
| C. pasteurianum (AF006034) | | | orfW | 210-731 | OrfX | 1-196 | orfY | 746-1177 |
| S. typhimurium (AF026270) | | | | | PduH | 8274-8645 | | |
| K. oxytoca (AF017781) | | | | | DdrB | 2063-2440 | | |
| K. oxytoca (AF051373) | | | | | | | | |
| | GENE FUNCTION: | | | | | | | |
| ORGANISM (GenBank | dehydratase, α | | dehydratase, β | | dehydratase, γ | | reactivation | |
| Reference) | gene | base pairs | gene | base pairs | gene | base pairs | gene | base pairs |
| K. pneumoniae (SEQ ID NO: 1) | dhaB1 | 7044-8711 | dhaB2 | 8724-9308 | dhaB3 | 9311-9736 | orfZ | 9749-11572 |
| K. pneumoniae (U30903) | dhaB1 | 3047-4714 | dhaB2 | 2450-2890 | dhaB3 | 2022-2447 | dhaB4 | 186-2009 |
| K. pneumoniae (U60992) | gldA | 121-1788 | gldB | 1801-2385 | GldC | 2388-2813 | gdrA | |
| C. freundii (U09771) | dhaB | 8556-10223 | dhaC | 10235-10819 | DhaE | 10822-11250 | orfZ | 11261-13072 |
| C. pasteurianum (AF051373) | dhaB | 84-1748 | dhaC | 1779-2318 | DhaE | 2333-2773 | orfZ | 2790-4598 |
| C. pasteurianum (AF006034) | | | | | | | | |
| S. typhimurium (AF026270) | pduC | 3557-5221 | pduD | 5232-5906 | PduE | 5921-6442 | pduG | 6452-8284 |
| K. oxytoca (AF017781) | | | | | | | ddrA | 241-2073 |
| K. oxytoca (AF051373) | pddA | 121-1785 | pddB | 1796-2470 | PddC | 2485-3006 | | |

The term “aldehyde dehydrogenase” and refers to a protein that catalyzes the conversion of an aldehyde to a carboxylic acid. Aldehyde dehydrogenases may use a redox cofactor such as NAD, NADP, FAD, or PQQ. Typical of aldehyde dehydrogenases is EC 1.2.1.3 (NAD-dependent); EC 1.2.1.4 (NADP-dependent); EC 1.2.99.3 (PQQ-dependent); or EC 1.2.99.7 (FAD-dependent). An example of an NADP-dependent aldehyde dehydrogenase is AIdB (SEQ ID NO:73), encoded by the E. coli gene aldB (SEQ ID NO:72). Examples of NAD-dependent aldehyde dehydrogenases include AIdA (SEQ ID NO:71), encoded by the E. coli gene aldA (SEQ ID NO:70); and AIdH (SEQ ID NO:75), encoded by the E. coli gene aldH (SEQ ID NO:74).

Genes that are Deleted:

The terms “NADH dehydrogenase II”, “NDH II” and “Ndh” refer to the type II NADH dehydrogenase, a protein that catalyzed the conversion of ubiquinone-8+NADH+H⁺ to ubiquinol-8+NAD⁺. Typical of NADH dehydrogenase II is EC 1.6.99.3. NADH dehydrogenase II is encoded by ndh in E. coli.

The terms “aerobic respiration control protein” and “ArcA” refer to a global regulatory protein. The aerobic respiration control protein is encoded by arcA in E. coli.

The terms “phosphogluconate dehydratase” and “Edd” refer to a protein that catalyzed the conversion of 6-phospho-gluconate to 2-keto-3-deoxy-6-phospho-gluconate+H₂O. Typical of phosphogluconate dehydratase is EC 4.2.1.12. Phosphogluconate dehydratase is encoded by edd in E. coli.

The terms “phosphocarrier protein HPr” and “PtsH” refer to the phosphocarrier protein encoded by ptsH in E. coli. The terms “phosphoenolpyruvate-protein phosphotransferase” and “Ptsl” refer to the phosphotransferase, EC 2.7.3.9, encoded by ptsl in E. coli. The terms “PTS system”, “glucose-specific IIA component”, and “Crr” refer to EC 2.7.1.69, encoded by crr in E. coli. PtsH, Ptsl, and Crr comprise the PTS system.

The term “phosphoenolpyruvate-sugar phosphotransferase system”, “PTS system”, or “PTS” refers to the phosphoenolpyruvate-dependent sugar uptake system.

The terms “methylglyoxal synthase” and “MgsA” refer to a protein that catalyzed the conversion of dihydroxy-acetone-phosphate to methyl-glyoxal+phosphate. Typical of methylglyoxal synthase is EC 4.2.3.3. Methylglyoxal synthase is encoded by mgsA in E. coli.

The term “1,3-propanediol dehydrogenase” refers to a protein that catalyzes the conversion of 3-hydroxypropionaldehyde to 1,3-propanediol. Such enzymes may utilize NAD, NADH or other redox cofactor. An example of an NADP-dependent 1,3-propanediol dehydrogenase is encoded by the yqhD gene in E. coli K-12 strains.

Genes Whose Expression has been Modified:

The terms “galactose-proton symporter” and “GaIP” refer to a protein that catalyses the transport of a sugar and a proton from the periplasm to the cytoplasm. D-glucose is a preferred substrate for GaIP. Galactose-proton symporter is encoded by galP in E. coli.

The terms “glucokinase” and “Glk” refer to a protein that catalyses the conversion of D-glucose+ATP to glucose-6-phosphate+ADP. Typical of glucokinase is EC 2.7.1.2. Glucokinase is encoded by glk in E. coli.

The terms “glyceraldehyde 3-phosphate dehydrogenase” and “GapA” refer to a protein that catalyses the conversion of glyceraldehyde 3-phosphate+phosphate+NAD⁺ to 3-phospho-D-glyceroyl-phosphate+NADH+H⁺. Typical of glyceraldehyde 3-phosphate dehydrogenase is EC 1.2.1.12. Glyceraldehyde 3-phosphate dehydrogenase is encoded by gapA in E. coli.

The terms “phosphoenolpyruvate carboxylase” and “Ppc” refer to a protein that catalyses the conversion of phosphoenolpyruvate+H₂O+CO₂ to phosphate+oxaloacetic acid. Typical of phosphoenolpyruvate carboxylase is EC 4.1.1.31. Phosphoenolpyruvate carboxylase is encoded by ppc in E. coli.

The term “YciK” refers to a putative enzyme encoded by yciK which is translationally coupled to btuR, the gene encoding Cob(I)alamin adenosyltransferase in Escherichia coli.

The term “cob(I)alamin adenosyltransferase” refers to an enzyme responsible for the transfer of a deoxyadenosyl moiety from ATP to the reduced corrinoid. Typical of cob(I)alamin adenosyltransferase is EC 2.5.1.17. Cob(I)alamin adenosyltransferase is encoded by the gene “btuR” (GenBank M21528) in Escherichia coli, “cobA” (GenBank L08890) in Salmonella typhimurium, and “cobO” (GenBank M62866) in Pseudomonas denitrificans.

### Additional Definitions:

The term “short 1.20 GI promoter” refers to SEQ ID NO:31. The term “short 1.5 GI promoter” refers to SEQ ID NO:28. The terms “short 1.6 GI promoter” and “short wild-type promoter” are used interchangeably and refer to SEQ ID NO:32.

The term “glycerol kinase” refers to a polypeptide responsible for an enzyme activity that catalyzes the conversion of glycerol and ATP to glycerol 3-phosphate and ADP. The high-energy phosphate donor ATP may be replaced by physiological substitutes (e.g., phosphoenolpyruvate). Glycerol kinase is encoded, for example, by GUT1 (GenBank U11583x19) and glpK (GenBank L19201) (see WO 9928480 and references).

The term “glycerol dehydrogenase” refers to a polypeptide responsible for an enzyme activity that catalyzes the conversion of glycerol to dihydroxyacetone (E.C. 1.1.1.6) or glycerol to glyceraldehyde (E.C. 1.1.1.72). A polypeptide responsible for an enzyme activity that catalyzes the conversion of glycerol to dihydroxyacetone is also referred to as a “dihydroxyacetone reductase”. Glycerol dehydrogenase may be dependent upon NAD (E.C. 1.1.1.6), NADP (E.C. 1.1.1.72), or other cofactors (e.g., E.C. 1.1.99.22). A NAD-dependent glycerol dehydrogenase is encoded, for example, by gldA (GenBank 000006) (see WO 9928480 and references therein).

Glycerol and diol dehydratases are subject to mechanism-based suicide inactivation by glycerol and some other substrates (Daniel et al., FEMS Microbiol. Rev. 22, 553 (1999)). The term “dehydratase reactivation factor” refers to those proteins responsible for reactivating the dehydratase activity. The terms “dehydratase reactivating activity”, “reactivating the dehydratase activity” or “regenerating the dehydratase activity” refers to the phenomenon of converting a dehydratase not capable of catalysis of a substrate to one capable of catalysis of a substrate or to the phenomenon of inhibiting the inactivation of a dehydratase or the phenomenon of extending the useful half-life of the dehydratase enzyme in vivo. Two proteins have been identified as being involved as the dehydratase reactivation factor (see WO 9821341 (U.S. Pat. No. 6,013,494) and references therein, which are herein incorporated by reference; Daniel et al., supra; Toraya and Mori, J. Biol. Chem. 274, 3372 (1999); and Tobimatsu et al., J. Bacteriol. 181, 4110 (1999)). Referring to Table 1, genes encoding one of the proteins include orfZ, dhaB4, gdrA, pduG and ddrA. Also referring to Table 1, genes encoding the second of the two proteins include orfX, orf2b, gdrB, pduH and ddrB.

The term “dha regulon” refers to a set of associated genes or open reading frames encoding various biological activities, including but not limited to a dehydratase activity, a reactivation activity, and a 1,3-propanediol oxidoreductase. Typically a dha regulon comprises the open reading frames dhaR, orfY, dhaT, orfX, orfW, dhaB1, dhaB2, dhaB3, and orfZ as described herein.

The terms “function” or “enzyme function” refer to the catalytic activity of an enzyme in altering the energy required to perform a specific chemical reaction. It is understood that such an activity may apply to a reaction in equilibrium where the production of either product or substrate may be accomplished under suitable conditions.

The terms “polypeptide” and “protein” are used interchangeably.

The terms “carbon substrate” and “carbon source” refer to a carbon source capable of being metabolized by host microorganisms of the present invention and particularly carbon sources selected from the group consisting of monosaccharides, oligosaccharides, polysaccharides, and one-carbon substrates or mixtures thereof. In one embodiment, the carbon source is glucose.

The term “renewably sourced carbon” refers to sources of carbon or carbohydrate that are derived from renewable agricultural feedstocks such as corn, soybeans, sugar cane and wheat, or other cellulosic or non-cellulosic feedstocks, rather than hydrocarbons that are considered non-renewable.

“Gene” refers to a nucleic acid fragment that expresses a specific protein, which may or may not include regulatory sequences preceding (5′ non-coding sequences) and following (3′ non-coding sequences) the coding sequence. “Native gene” or “wild type gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

The term “genetic construct” refers to a nucleic acid fragment that encodes for expression of one or more specific proteins. In the gene construct the gene may be native, chimeric, or foreign in nature. Typically a genetic construct will comprise a “coding sequence”. A “coding sequence” refers to a DNA sequence that codes for a specific amino acid sequence.

“Promoter” or “Initiation control regions” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”.

The term “expression”, as used herein, refers to the transcription and stable accumulation of sense (mRNA) or antisense RNA derived from a gene. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts or fragments capable of suppressing the expression of identical or substantially similar foreign or endogenous genes (U.S. Pat. No. 5,231,020).

The term “transformation” as used herein, refers to the transfer of a nucleic acid fragment into a host organism, resulting in genetically stable inheritance. The transferred nucleic acid may be in the form of a plasmid maintained in the host cell, or some transferred nucleic acid may be integrated into the genome of the host cell. Host organisms containing the transformed nucleic acid fragments are referred to as “transgenic” or “recombinant” or “transformed” organisms.

The terms “plasmid” and “vector” as used herein, refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “selectable marker” means an identifying factor, usually an antibiotic or chemical resistance gene, that is able to be selected for based upon the marker gene's effect, i.e., resistance to an antibiotic, wherein the effect is used to track the inheritance of a nucleic acid of interest and/or to identify a cell or organism that has inherited the nucleic acid of interest.

As used herein the term “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

### Construction of Recombinant Organisms

Recombinant organisms containing the necessary genes that will encode the enzymatic pathway for the conversion of a carbon substrate to 3-HP may be constructed using techniques well known in the art. Genes encoding glycerol-3-phosphate dehydrogenase (GPD1), glycerol 3-phosphatase (GPP2), glycerol dehydratase (dhaB1, dhaB2, and dhaB3), dehydratase reactivation factor (orfZ and orfX) and aldehyde dehydrogenase (e.g., aldA, aldB, or aldH) may be isolated from a native host such as Klebsiella, Saccharomyces or E. coli and used to transform host strains such as E. coli DH5α, ECL707, AA200, or KLP23.

### Isolation of Genes

Methods of obtaining desired genes from a bacterial genome are common and well known in the art of molecular biology. For example, if the sequence of the gene is known, suitable genomic libraries may be created by restriction endonuclease digestion and may be screened with probes complementary to the desired gene sequence. Once the sequence is isolated, the DNA may be amplified using standard primer directed amplification methods such as polymerase chain reaction (PCR) (U.S. Pat. No. 4,683,202) to obtain amounts of DNA suitable for transformation using appropriate vectors.

Alternatively, cosmid libraries may be created where large segments of genomic DNA (35-45 kb) may be packaged into vectors and used to transform appropriate hosts. Cosmid vectors are unique in being able to accommodate large quantities of DNA. Generally cosmid vectors have at least one copy of the cos DNA sequence which is needed for packaging and subsequent circularization of the foreign DNA. In addition to the cos sequence these vectors will also contain an origin of replication such as ColE1 and drug resistance markers such as a gene resistant to ampicillin or neomycin. Methods of using cosmid vectors for the transformation of suitable bacterial hosts are well described in Sambrook, J., Fritsch, E. F. and Maniatis, T. Molecular Cloning: A Laboratory Manual, Second Edition, Cold Spring Harbor Laboratory Press, Cold Spring Harbor (1989).

Typically to clone cosmids, foreign DNA is isolated using the appropriate restriction endonucleases and ligated, adjacent to the cos region of the cosmid vector using the appropriate ligases. Cosmid vectors containing the linearized foreign DNA are then reacted with a DNA packaging vehicle such as bacteriophage. During the packaging process the cos sites are cleaved and the foreign DNA is packaged into the head portion of the bacterial viral particle. These particles are then used to transfect suitable host cells such as E. coli. Once injected into the cell, the foreign DNA circularizes under the influence of the cos sticky ends. In this manner large segments of foreign DNA can be introduced and expressed in recombinant host cells.

Isolation and Cloning of Genes Encoding Glycerol Dehydratase (dhaB1, dhaB2, and dhaB3), and Dehydratase Reactivating Factors (orfZ and orfX)

Cosmid vectors and cosmid transformation methods may be used within the context of the present invention to clone large segments of genomic DNA from bacterial genera known to possess genes capable of processing glycerol to 3-hydroxypropionaldehyde. Specifically, genomic DNA from K. pneumoniae may be isolated by methods well known in the art and digested with the restriction enzyme Sau3A for insertion into a cosmid vector Supercos 1 and packaged using GigapackII packaging extracts. Following construction of the vector E. coli XL1 Blue MR cells may be transformed with the cosmid DNA. Transformants may be screened for the ability to convert glycerol to 3-hydroxypropionaldehyde by growing the cells in the presence of glycerol and analyzing the media for the presence of 3-hydroxypropionaldehyde or derivatives such as PDO or 3-HP.

Although the instant invention utilizes the isolated genes from within a Klebsiella cosmid, alternate sources of dehydratase genes and dehydratase reactivation factor genes include, but are not limited to, Citrobacter, Clostridia and Salmonella species.

### Genes Encoding G3PDH and G3P Phosphatase

The present invention provides genes suitable for the expression of G3PDH and G3P phosphatase activities in a host cell.

Genes encoding G3PDH are known. For example, GPD1 has been isolated from Saccharomyces cerevisiae (Wang et al., J. Bact. 176, 7091-7095 (1994)). Similarly, G3PDH activity has also been isolated from Saccharomyces cerevisiae encoded by GPD2 (Eriksson et al., Mol. Microbiol. 17, 95 (1995)).

For the purposes of the present invention it is contemplated that any gene encoding a polypeptide responsible for NAD-dependent G3PDH activity is suitable wherein that activity is capable of catalyzing the conversion of dihydroxyacetone phosphate (DHAP) to glycerol 3-phosphate (G3P). Further, it is contemplated that any gene encoding the amino acid sequence of NAD-dependent G3PDH′s corresponding to the genes DAR1, GPD1, GPD2, GPD3, and gpsA will be functional in the present invention wherein that amino acid sequence may encompass amino acid substitutions, deletions or additions that do not alter the function of the enzyme. The skilled person will appreciate that genes encoding G3PDH isolated from other sources will also be suitable for use in the present invention.

Genes encoding G3P phosphatase are known. For example, GPP2 has been isolated from Saccharomyces cerevisiae (Norbeck et al., J. Biol. Chem. 271, 13875 (1996)). For the purposes of the present invention, any gene encoding a G3P phosphatase activity is suitable for use in the method wherein that activity is capable of catalyzing the conversion of glycerol 3-phosphate plus H₂O to glycerol plus inorganic phosphate. Further, any gene encoding the amino acid sequence of G3P phosphatase corresponding to the genes GPP2 and GPP1 will be functional in the present invention including any amino acid sequence that encompasses amino acid substitutions, deletions or additions that do not alter the function of the G3P phosphatase enzyme. The skilled person will appreciate that genes encoding G3P phosphatase isolated from other sources will also be suitable for use in the present invention.

### Genes Encoding Aldehyde Dehydrogenase

Genes encoding aldehyde dehydrogenase are known. Suitable examples include, but are not limited to, aldA (SEQ ID NO:70), aldB (SEQ ID NO:72), and aldH (SEQ ID NO:74). For the purposes of the present invention, any gene encoding an aldehyde dehydrogenase is suitable for use herein, wherein that activity is capable of catalyzing the conversion of 3-hydroxypropionaldehyde to 3-HP. Further, any gene encoding the amino acid sequence of aldehyde dehydrogenase corresponding to the genes aldA, aldB, or aldH will be functional in the present invention including any amino acid sequence that encompasses amino acid substitutions, deletions or additions that do not alter the function of the aldehyde dehydrogenase enzyme. The skilled person will appreciate that genes encoding aldehyde dehydrogenase isolated from other sources will also be suitable for use in the present invention.

### Host Cells

Suitable host cells for the recombinant production of 3-HP may be either prokaryotic or eukaryotic and will be limited only by the host cell ability to express the active enzymes for the 3-HP pathway. Suitable host cells will be microorganisms from genera such as Citrobacter, Enterobacter, Clostridium, Klebsiella, Aerobacter, Lactobacillus, Aspergillus, Saccharomyces, Schizosaccharomyces, Zygosaccharomyces, Pichia, Kluyveromyces, Candida, Hansenula, Debaryomyces, Mucor, Torulopsis, Methylobacter, Escherichia, Salmonella, Bacillus, Streptomyces, and Pseudomonas. Preferred in the present invention are Escherichia coli, Escherichia blattae, Klebsiella species, Citrobacter species, and Aerobacter species. Most preferred is E. coli (KLP23 (WO 2001012833 A2), RJ8.n (ATCC PTA-4216), E. coli: FMP′::Km (ATCC PTA4732), MG 1655 (ATCC 700926)).

### Vectors and Expression Cassettes

A variety of vectors and transformation and expression cassettes are suitable for the cloning, transformation and expression of G3PDH, G3P phosphatase, glycerol dehydratase, dehydratase reactivation factor, and aldehyde dehydrogenase into a suitable host cell. Suitable vectors will be those which are compatible with the microorganism employed. Suitable vectors can be derived, for example, from a bacterium, a virus (such as bacteriophage T7 or a M-13 derived phage), a cosmid, a yeast or a plant. Protocols for obtaining and using such vectors are known to those in the art (Sambrook et al., supra).

Initiation control regions, or promoters, which are useful to drive expression of the G3PDH and G3P phosphatase genes (DAR1 and GPP2, respectively), and aldehyde dehydrogenase genes in the desired host cell are numerous and familiar to those skilled in the art. Virtually any promoter capable of driving these genes is suitable for the present invention including but not limited to CYC1, HIS3, GAL1, GAL10, ADH1, PGK, PHO5, GAPDH, ADC1, TRP1, URA3, LEU2, ENO, and TPI (useful for expression in Saccharomyces species); AOX1 (useful for expression in Pichia species); and lac, trp, XP_{L}, XP_{R}, T7, tac, and trc (useful for expression in E. coli).

Termination control regions may also be derived from various genes native to the preferred hosts. Optionally, a termination site may be unnecessary; however, it is most preferred if included.

For effective expression of the instant enzymes, DNA encoding the enzymes are linked operably through initiation codons to selected expression control regions such that expression results in the formation of the appropriate messenger RNA.

Particularly useful in the present invention are the vectors pSYCO101, pSYCO103, pSYCO106, and pSYCO109. The essential elements are derived from the dha regulon isolated from Klebsiella pneumoniae and from Saccharomyces cerevisiae. Each contains the open reading frames dhaB1 , dhaB2, dhaB3, dhaX (SEQ ID NO:69), orfX, DAR1, and GPP2 arranged in three separate operons, nucleotide sequences of which are given in SEQ ID NO:54, SEQ ID NO:55, SEQ ID NO:56, and SEQ ID NO:57, respectively. The differences between the vectors are illustrated in the chart below [the prefix “p-” indicates a promoter; the open reading frames contained within each “( )” represent the composition of an operon]:

pSYCO101 (SEQ ID NO:54):

p-trc (Dar1_GPP2) in opposite orientation compared to the other 2 pathway operons,

p-1.6 long GI (dhaB1_dhaB2_dhaB3_dhaX), and

p-1.6 long GI (orfY_orfX_orfW). pSYCO103 (SEQ ID NO:55):

p-trc (Dar1_GPP2) same orientation compared to the other 2 pathway operons,

p-1.5 long GI (dhaB1_dhaB2_dhaB3_dhaX), and

p-1.5 long GI (orfY_orfX_orfW). pSYCO106 (SEQ ID NO:56):

p-trc (Dar1_GPP2) same orientation compared to the other 2 pathway operons,

p-1.6 long GI (dhaB1_dhaB2_dhaB3_dhaX), and

p-1.6 long GI (orfY_orfX_orfW). pSYCO109 (SEQ ID NO:57):

p-trc (Dar1_GPP2) same orientation compared to the other 2 pathway operons,

p-1.6 long GI (dhaB1_dhaB2_dhaB3_dhaX), and

p-1.6 long GI (orfY_orfX).

### Transformation of Suitable Hosts and Expression of Genes for the Production of 3-HP

Once suitable cassettes are constructed they are used to transform appropriate host cells. Introduction of the cassette containing the genes encoding G3PDH, G3P phosphatase, glycerol dehydratase, dehydratase reactivation factor, and aldehyde dehydrogenase into the host cell may be accomplished by known procedures such as by transformation (e.g., using calcium-permeabilized cells, electroporation), or by transfection using a recombinant phage virus (Sambrook et al., supra).

In the present invention cassettes may be used to transform the E. coli as fully described in the GENERAL METHODS and EXAMPLES.

### Mutants

In addition to the cells exemplified, it is contemplated that the present method will be able to make use of cells having single or multiple mutations specifically designed to enhance the production of 3-HP. Cells that normally divert a carbon feed stock into non-productive pathways, or that exhibit significant catabolite repression could be mutated to avoid these phenotypic deficiencies. For example, many wild-type cells are subject to catabolite repression from glucose and by-products in the media and it is contemplated that mutant strains of these wild-type organisms, capable of 3-HP production that are resistant to glucose repression, would be particularly useful in the present invention.

Methods of creating mutants are common and well known in the art. For example, wild-type cells may be exposed to a variety of agents such as radiation or chemical mutagens and then screened for the desired phenotype. When creating mutations through radiation either ultraviolet (UV) or ionizing radiation may be used. Suitable short wave UV wavelengths for genetic mutations will fall within the range of 200 nm to 300 nm where 254 nm is preferred. UV radiation in this wavelength principally causes changes within nucleic acid sequence from guanidine and cytosine to adenine and thymidine. Since all cells have DNA repair mechanisms that would repair most UV induced mutations, agents such as caffeine and other inhibitors may be added to interrupt the repair process and maximize the number of effective mutations. Long wave UV mutations using light in the 300 nm to 400 nm range are also possible but are generally not as effective as the short wave UV light unless used in conjunction with various activators such as psoralen dyes that interact with the DNA.

Mutagenesis with chemical agents is also effective for generating mutants and commonly used substances include chemicals that affect nonreplicating DNA such as HNO₂ and NH₂OH, as well as agents that affect replicating DNA such as acridine dyes, notable for causing frameshift mutations. Specific methods for creating mutants using radiation or chemical agents are well documented in the art. See, for example, Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol. 36, 227 (1992).

After mutagenesis has occurred, mutants having the desired phenotype may be selected by a variety of methods. Random screening is most common where the mutagenized cells are selected for the ability to produce the desired product or intermediate. Alternatively, selective isolation of mutants can be performed by growing a mutagenized population on selective media where only resistant colonies can develop. Methods of mutant selection are highly developed and well known in the art of industrial microbiology. See for example Brock, Supra; DeMancilha et al., Food Chem. 14, 313 (1984).

In addition to the methods for creating mutants described above, selected genes involved in converting carbon substrate to 3-HP may be up-regulated or down-regulated by a variety of methods which are known to those skilled in the art. It is well understood that up-regulation or down-regulation of a gene refers to an alteration in the activity of the protein encoded by that gene relative to a control level of activity, for example, by the activity of the protein encoded by the corresponding (or non-altered) wild-type gene.

### Up-Regulation:

Specific genes involved in an enzyme pathway may be up-regulated to increase the activity of their encoded function(s). For example, additional copies of selected genes may be introduced into the host cell on multicopy plasmids such as pBR322. Such genes may also be integrated into the chromosome with appropriate regulatory sequences that result in increased activity of their encoded functions. The target genes may be modified so as to be under the control of non-native promoters or altered native promoters. Endogenous promoters can be altered in vivo by mutation, deletion, and/or substitution.

### Down-Regulation:

Alternatively, it may be useful to reduce or eliminate the expression of certain genes relative to a given activity level. For the purposes of this invention, it is useful to distinguish between reduction and elimination. The terms “down regulation” and “down-regulating” of a gene refers to a reduction, but not a total elimination, of the activity of the encoded protein. Methods of down-regulating and disrupting genes are known to those of skill in the art.

Down-regulation can occur by deletion, insertion, or alteration of coding regions and/or regulatory (promoter) regions. Specific down regulations may be obtained by random mutation followed by screening or selection, or, where the gene sequence is known, by direct intervention by molecular biology methods known to those skilled in the art. A particularly useful, but not exclusive, method to effect down-regulation is to alter promoter strength.

### Disruption:

Disruptions of genes may occur, for example, by 1) deleting coding regions and/or regulatory (promoter) regions, 2) inserting exogenous nucleic acid sequences into coding regions and/regulatory (promoter) regions, and 3) altering coding regions and/or regulatory (promoter) regions (for example, by making DNA base pair changes). Such changes would either prevent expression of the protein of interest or result in the expression of a protein that is non-functional. Specific disruptions may be obtained by random mutation followed by screening or selection, or, in cases where the gene sequences in known, specific disruptions may be obtained by direct intervention using molecular biology methods know to those skilled in the art. A particularly useful method is the deletion of significant amounts of coding regions and/or regulatory (promoter) regions.

Methods of altering recombinant protein expression are known to those skilled in the art, and are discussed in part in Baneyx, Curr. Opinion Biotech. (1999) 10:411; Ross, et al., J Bacteriol. (1998) 180:5375; deHaseth, et al., J. Bacteriol. (1998) 180:3019; Smolke and Keasling, Biotech. And Bioengineeering (2002) 80:762; Swartz, Curr. Opinions Biotech.(2001) 12:195; and Ma, et al., J. Bacteriol. (2002) 184:5733.

### Alterations in the 3-HP Production Pathway

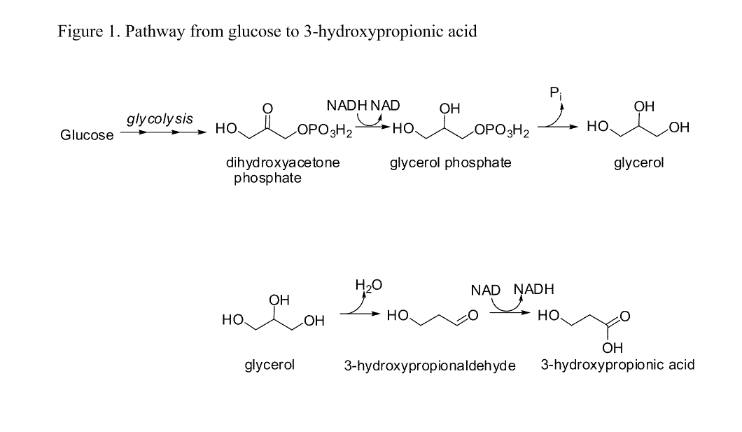

Representative Enzyme Pathway. The production of 3-HP from glucose can be accomplished by the following series of steps, as shown in FIG. 1. This series is representative of a number of pathways known to those skilled in the art. Glucose is converted in a series of steps by enzymes of the glycolytic pathway to dihydroxyacetone phosphate (DHAP). The remainder of the pathway comprises the following substrate to product conversions:
a) dihydroxyacetone phosphate to glycerol phosphate, catalyzed by glycerol-3-phosphate dehydrogenase,
b) glycerol phosphate to glycerol, catalyzed by glycerol 3-phosphatase,
c) glycerol to 3-hydroxypropionaldehyde, catalyzed by glycerol dehydratase, and
d) 3-hydroxypropionaldehyde to 3-HP, catalyzed by aldehyde dehydrogenase.
   Mutations and Transformations that Affect Carbon Channeling.

A variety of mutant microorganisms comprising variations in the 3-HP production pathway will be useful in the present invention. Mutations which block alternate pathways for intermediates of the 3-HP production pathway would also be useful to the present invention. For example, the elimination of glycerol kinase prevents glycerol, formed from G3P by the action of G3P phosphatase, from being re-converted to G3P at the expense of ATP. Also, the elimination of glycerol dehydrogenase (for example, gldA) prevents glycerol, formed from DHAP by the action of NAD-dependent glycerol-3-phosphate dehydrogenase, from being converted to dihydroxyacetone. Mutations can be directed toward a structural gene so as to impair or improve the activity of an enzymatic activity or can be directed toward a regulatory gene, including promoter regions and ribosome binding sites, so as to modulate the expression level of an enzymatic activity.

It is thus contemplated that transformations and mutations can be combined so as to control particular enzyme activities for the enhancement of 3-HP production. Thus, it is within the scope of the present invention to anticipate modifications of a whole cell catalyst which lead to an increased production of 3-HP.

In one embodiment, the present invention utilizes a preferred pathway for the production of 3-HP from a sugar substrate where the carbon flow moves from glucose to DHAP, G3P, glycerol, 3-HPA, and finally to 3-HP. The present production strains may be engineered to maximize the metabolic efficiency of the pathway by incorporating various deletion mutations that prevent the diversion of carbon to non-productive compounds. Glycerol may be diverted from conversion to 3HPA by transformation to either DHA or G3P via glycerol dehydrogenase or glycerol kinase as discussed above. Accordingly, the present production strains may contain deletion mutations in the gldA and glpK genes. Similarly DHAP may be diverted to 3-PG by triosephosphate isomerase, thus the present production microorganism may also contain a deletion mutation in this gene. The present method additionally incorporates a glycerol dehydratase enzyme for the conversion of glycerol to 3-hydroxypropionaldehyde, which functions in concert with the reactivation factor, encoded by orfX and orfZ of the dha regulon.

In one embodiment, the endogenous yqhD gene (SEQ ID NO:76) is deleted from an E. coli host strain comprising the 3-HP production pathway. This deletion prevents conversion of 3-hydroxypropionaldehye to 1,3-propanediol.

### Media and Carbon Substrates

Fermentation media in the present invention must contain suitable carbon substrates. Suitable substrates may include but are not limited to monosaccharides such as glucose and fructose and oligosaccharides such as lactose or sucrose.

In the present invention, the preferred carbon substrate is glucose. In addition to an appropriate carbon source, fermentation media must contain suitable minerals, salts, cofactors, buffers and other components, known to those skilled in the art, suitable for the growth of the cultures and promotion of the enzymatic pathway necessary for 3-HP production. Particular attention is given to Co(II) salts and/or vitamin B₁₂ or precursors thereof.

Adenosyl-cobalamin (coenzyme B₁₂) is an essential cofactor for dehydratase activity. Synthesis of coenzyme B₁₂ is found in prokaryotes, some of which are able to synthesize the compound de novo, for example, Escherichia blattae, Klebsiella species, Citrobacter species, and Clostridium species, while others can perform partial reactions. E. coli, for example, cannot fabricate the corrin ring structure, but is able to catalyze the conversion of cobinamide to corrinoid and can introduce the 5′-deoxyadenosyl group. Thus, it is known in the art that a coenzyme B₁₂ precursor, such as vitamin B₁₂, need be provided in E. coli fermentations.

Vitamin B₁₂ additions to E. coli fermentations may be added continuously, at a constant rate or staged as to coincide with the generation of cell mass, or may be added in single or multiple bolus additions. Preferred ratios of vitamin B₁₂ (mg) fed to cell mass (OD550) are from 0.06 to 0.60. Most preferred ratios of vitamin B₁₂ (mg) fed to cell mass (OD550) are from 0.12 to 0.48.

Although vitamin B₁₂ is added to the transformed E. coli of the present invention it is contemplated that other microorganisms, capable of de novo B₁₂ biosynthesis will also be suitable production cells and the addition of B₁₂ to these microorganisms will be unnecessary.

### Culture Conditions:

Typically cells are grown at 35° C. in appropriate media. Preferred growth media in the present invention are common commercially prepared media such as Luria Bertani (LB) broth, Sabouraud Dextrose (SD) broth or Yeast medium (YM) broth. Other defined or synthetic growth media may also be used and the appropriate medium for growth of the particular microorganism will be known by someone skilled in the art of microbiology or fermentation science. The use of agents known to modulate catabolite repression directly or indirectly, e.g., cyclic adenosine 2′:3′-monophosphate, may also be incorporated into the reaction media. Similarly, the use of agents known to modulate enzymatic activities (e.g., methyl viologen) that lead to enhancement of 1,3-propanediol production may be used in conjunction with or as an alternative to genetic manipulations.

Suitable pH ranges for the fermentation are between pH 5.0 to pH 9.0, where pH 6.0 to pH 8.0 is preferred as the initial condition.

Reactions may be performed under aerobic or anaerobic conditions where aerobic, anoxic, or anaerobic conditions are preferred based on the requirements of the microorganism.

Fed-batch fermentations may be performed with carbon feed, for example, glucose, limited or excess.

### Batch and Continuous Fermentations:

The present process employs a batch method of fermentation.

Classical batch fermentation is a closed system where the composition of the medium is set at the beginning of the fermentation and is not subject to artificial alterations during the fermentation. Thus, at the beginning of the fermentation the medium is inoculated with the desired microorganism or microorganisms, and fermentation is permitted to occur adding nothing to the system. Typically, however, “batch” fermentation is batch with respect to the addition of carbon source and attempts are often made at controlling factors such as pH and oxygen concentration. In batch systems the metabolite and biomass compositions of the system change constantly up to the time the fermentation is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of end product or intermediate.

A variation on the standard batch system is the Fed-Batch system. Fed-Batch fermentation processes are also suitable in the present invention and comprise a typical batch system with the exception that the substrate is added in increments as the fermentation progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch fermentations are common and well known in the art and examples may be found in Brock, supra.

Although the present invention is performed in batch mode it is contemplated that the method would be adaptable to continuous fermentation methods. Continuous fermentation is an open system where a defined fermentation media is added continuously to a bioreactor and an equal amount of conditioned media is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity, is kept constant. Continuous systems strive to maintain steady state growth conditions and thus the cell loss due to media being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

It is contemplated that the present invention may be practiced using batch, fed-batch or continuous processes and that any known mode of fermentation would be suitable. Additionally, it is contemplated that cells may be immobilized on a substrate as whole cell catalysts and subjected to fermentation conditions for 3-HP production.

### Identification and Purification of 3-HP:

Methods for the purification of 3-HP from fermentation media are known in the art. For example, 3-HP can be obtained from cell media by subjecting the reaction mixture to column chromatography.

3-HP may be identified directly by submitting the media to high pressure liquid chromatography (HPLC) analysis. Preferred in the present invention is a method where fermentation media is analyzed on an analytical ion exchange column using a mobile phase of 0.01 N sulfuric acid in an isocratic fashion.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### General Methods

Standard recombinant DNA and molecular cloning techniques described in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following Examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials described for the growth and maintenance of bacterial cells may be obtained from Aldrich Chemicals (Milwaukee, Wis.), BD Diagnostic Systems (Sparks, Md.), Life Technologies (Rockville, Md.), or Sigma Chemical Company (St. Louis, Mo.).

The meaning of abbreviations is as follows: “s” means second(s), “min” means minute(s), “h” means hour(s), “nm” means nanometers, “μL” means microliter(s), “mL” means milliliter(s), “L” means liter(s), “nm” means nanometers, “mM” means millimolar, “M” means molar, “mmol” means millimole(s), “μmol” means micromole(s)”, “g” means gram(s), “μg” means microgram(s) and “rpm” means revolutions per minute.

### Example 1

### Prophetic

### Construction of 3-Hydroxypropionic Acid Producing Strains

Three endogenous E. coli genes encoding aldehyde dehydrogenases, specifically, aldA given as SEQ ID NO:70, aldB given as SEQ ID NO:72, and aldH given as SEQ ID NO:74, are amplified from E. coli strain MG1655 genomic DNA, which may be obtained from the American Type Culture Collection (ATCC, Manassas, Va.), in separate PCR reactions using primer pairs: Afor (SEQ ID NO:77) and Arev (SEQ ID NO:78); Bfor (SEQ ID NO:79) and Brev (SEQ ID NO:80); and Hfor (SEQ ID NO:81) and Hrev (SEQ ID NO:82); respectively. These primers result in the presence of HindIII recognition sites at each end of the open reading frames in the amplified products. The resulting amplification products (1440, 1539 and 1488 base pairs, respectively) are digested with HindIII and ligated with similarly digested pKK223-3 vector [Brosius J and Holy A (1984) Pro. Natl. Acad. Sci. USA 22:6929-33]. The ligation mixture is used to transform E. coli strain TOP10 (Invitrogen, Carlsbad, Calif.), and the transformants are selected by growth on LB (Luria-Bertani) agar containing 100 μg/mL ampicillin. Individual colonies are picked and grown in overnight cultures (5 mL of LB broth containing 100 μg/mL ampicillin), from which plasmid DNA is isolated. The plasmid DNA is sequenced to identify clones in which the open reading frames are properly inserted and oriented such that gene transcription will be controlled by the tac promoter. These plasmids are designated: pKKaldA, pKKaldB and pKKaldH, and are subsequently used to transform E. coli strain TT/pSYCO109 (described in U.S. Pat. No. 7,371,558, Example 14). Transformants are selected by growth on LB agar containing 50 μg/mL spectinomycin and 100 μg/mL ampicillin. The resulting strains are designated herein as TT/pSYCO109/pKKaldA, TT/pSYCO109/pKKaldB, and TT/pSYCO109/pKKaldH, respectively. The TT/pSYCO109 strain is also transformed with plasmid pKK223-3 to serve as a control, giving strain TT/pSYCO109/pKK223-3.

### Example 2

### Prophetic

### Production of 3-Hydroxypropionic Acid by Transformed Strains

All 4 strains described in Example 1 (i.e., TT/pSYCO109/pKKaldA, TT/pSYCO109/pKKaldB, TT/pSYCO109/pKKaldH and TT/pSYCO109/pKK223-3) are grown overnight at 34° C. with shaking (250 rpm) in 5 mL of LB broth containing 50 μg/mL spectinomycin and 100 μg/mL ampicillin. These overnight cultures are diluted into TM3 medium containing 10 g/L glucose to an optical density of 0.01 units measured at 550 nm. TM3 is a minimal medium containing 13.6 g/L KH₂PO₄, 2.04 g/L citric acid dihydrate, 2 g/L magnesium sulfate heptahydrate, 0.33 g/L ferric ammonium citrate, 0.5 g/L yeast extract, 3 g/L ammonium sulfate, 0.2 g/L CaCl₂.2H₂O, 0.03 g MnSO₄.H₂O, 0.01 g/L NaCl, 1 mg/L FeSO₄.7H₂O, 1 mg/L, CoCl₂.6H₂O, 1 mg/L ZnSO₄.7H₂O, 0.1 mg/L CuSO₄.5H₂O, 0.1 mg/L H₃BO₄, 0.1 mg/L NaMoO₄.2H₂O, 0.1 mg/L vitamin B₁₂ and sufficient NH₄OH to provide a final pH of 6.8. The antibiotics spectinomycin (50 pg/mL) and ampicillin (100 μg/mL) are added to select for plasmid maintenance. The cultures are incubated at 34° C. with shaking (225 rpm) for 48 hours. Aliquots are removed at 0, 12, 24, 36 and 48 hours after inoculation, and the concentrations of glucose, glycerol and 3-hydroxypropionic acid in the broth are determined by high performance liquid chromatography. Chromatographic separation is achieved using a Shodex SH1011 column (Showa Denko America Inc., New York, N.Y.) with an isocratic mobile phase of 0.01 N H₂SO₄ in water at a flow rate of 0.5 mL/min. Eluted compounds are quantified by refractive index and UV detection with reference to a standard curve prepared from commercially purchased pure compounds diluted to known concentrations in the TM3 medium. Quantification is further confirmed by LC/MS (liquid chromatography/mass spectrometry) analysis of samples. At these conditions, it is expected that all three strains containing aldehyde dehydrogenase genes on the pKK plasmids (i.e., TT/pSYCO109/pKKaldA, TT/pSYCO109/pKKaldB, and TT/pSYCO109/pKKaldH), will produce more 3-hydroxypropionic acid than the control strain TT/pSYCO109/pKK223-3.

### Example 3

### Prophetic

### Construction of Improved 3-Hydroxypropionic Acid Producing Strains

A deletion of the yqhD gene (given as SEQ ID NO:76), which encodes a nonspecific alcohol dehydrogenase, is made in E. coli strain TT/pSYCO109 (described in U.S. Pat. No. 7,371,558, Example 14) by P1 transduction. The donor strain is E. coli BW25113 with a deletion of yqhD marked by KanR from the Keio collection (T. Baba et al. 2006. Mol. Syst. Biol. 2, 2006.0008). P1vir is grown on the donor strain and the phage stock is used for transduction of TT/pSYCO109, selecting for kanamcyin and spectinomycin resistance (J. Miller, Experiments in Molecular Genetics, 1972, Cold Spring Harbor Laboratory Press, Cold Spring Harbor, N.Y.). Following single colony purification, the resultant kanamycin and spectinomycin resistant strain is named TTΔyqhD::Kan/pSYCO109. Strain TTΔyqhD::Kan/pSYCO109 is transformed separately with pKKaldA, pKKaldB and pKKaldH. Transformants are selected by growth on LB agar containing 50 μg/mL spectinomycin and 100 μg/mL ampicillin. The resultant strains, which are resistant to kanamycin, ampicillin and spectinomycin, are designated herein as TTΔyqhD::Kan/pSYCO109/pKKaldA, TTΔyqhD::Kan/pSYCO109/pKKaldB, and TTΔyqhD::Kan/pSYCO109/pKKaldH. These three strains and TT/pSYCO109/pKKaldA, TT/pSYCO109/pKKaldB, TT/pSYCO109/pKKaldH are grown in 5 mL cultures of LB broth containing 50 μg/mL spectinomycin and 100 μg/mL ampicillin at 37° C., 250 rpm. These overnight cultures are diluted into TM3 medium containing 10 g/L glucose to an optical density of 0.01 units measured at 550 nm, as described in Example 2. The cultures are incubated at 34° C. with shaking (225 rpm) for 48 hours. Aliquots are removed at 0, 12, 24, 36 and 48 hours after inoculation, and the concentrations of glucose, glycerol and 3-hydroxypropionic acid in the broth are determined by high performance liquid chromatography and confirmed using LC/MS, as described in Example 2. At these conditions, it is expected that strain TTΔyqhD::Kan/pSYCO109/pKKaldA will produce more 3-hydroxypropionic acid than TT/pSYCO109/pKKaldA. Likewise, it is expected that TTΔyqhD::Kan/pSYCO109/pKKaldB will produce more 3-hydroxypropionic acid than TT/pSYCO109/pKKaldB, and TTΔyqhD::Kan/pSYCO109/pKKaldH will produce more 3-hydroxypropionic acid than TT/pSYCO109/pKKaldH.

What is claimed is:

1. An E. coli strain comprising:
a) an exogenous gene encoding a glycerol-3-phosphate dehydrogenase;
b) an exogenous gene encoding a glycerol 3-phosphatase;
c) exogenous genes encoding alpha, beta, and gamma subunits of glycerol dehydratase; and
d) an overexpression of a gene encoding an aldehyde dehydrogenase;
whereby said E. coli strain is capable of bioconverting a suitable carbon source to 3-hydroxypropionic acid.

2. The E. coli strain of clam 1 wherein the aldehyde dehydrogenase has an amino acid sequence selected from the group consisting of SEQ ID NO:71, SEQ ID NO:73, and SEQ ID NO:75.

3. The E. coli strain of claim 1 further comprising a deletion of an endogenous gene encoding a 1,3-propanediol dehydrogenase.

4. The E. coli strain of claim 3 wherein the endogenous 1,3-propanediol dehydrogenase gene has a nucleotide sequence as set forth in SEQ ID NO:76.

5. The E. coli strain of claim 1 further comprising:
e) a disrupted endogenous phosphoenolpyruvate-glucose phosphotransferase system comprising one or more of:
i) a genetically disrupted endogenous ptsH gene preventing expression of active phosphocarrier protein;
ii) a genetically disrupted endogenous ptsl gene preventing expression of active phosphoenolpyruvate-protein phosphotransferase; and
iii) a genetically disrupted endogenous crr gene preventing expression of active glucose-specific IIA component;
f) a genetically up regulated endogenous galP gene encoding active galactose-proton symporter, said up regulation resulting in an increased galactose-proton symporter activity; wherein the up regulation is produced by (a) by introducing additional copies of said gene into host cell followed by integration or (b) by replacing native regulatory sequence with strong non-native promoter or altered native promoter;
g) a genetically up regulated endogenous glk gene encoding active glucokinase, said up regulation resulting in an increased glucokinase activity; wherein the up regulation is produced by a) by introducing additional copies of said gene into host cell followed by integration or b) by replacing native regulatory sequence with strong non-native promoter or altered native promoter, and
h) a genetically down regulated endogenous gapA gene encoding active glyceraldehyde-3-phosphate dehydrogenase, said down regulation resulting in a reduced glyceraldehyde-3-phosphate dehydrogenase activity.

6. The E. coli strain of any of claim 1 or 5 further comprising a genetically disrupted endogenous arcA gene preventing expression of active aerobic respiration control protein.

7. The E. coli strain of claim 1 wherein the glycerol-3-phosphate dehydrogenase has an amino acid sequence as set forth in SEQ ID NO:59.

8. The E. coli strain of claim 1 wherein the genes encoding the alpha, beta, and gamma subunits of glycerol dehydratase have the nucleotide sequences as set forth in SEQ ID NO:66, SEQ ID NO:67, and SEQ ID NO:68.

9. A method for biologically producing 3-hydroxypropionic acid comprising contacting the strain of claim 1 with a suitable carbon substrate.

10. The method of claim 9 wherein said suitable carbon substrate is glucose.

11. A composition comprising the 3-hydroxypropionic acid produced from the method of claim 9 or 10, wherein said 3-hydroxyproprionic acid comprises renewably sourced carbon.

12. A composition comprising an intermediate of the 3-hydroxypropionic acid produced form the method of claim 9 or 10, wherein said intermediate comprises renewably sourced carbon.

13. The composition of claim 12, wherein said intermediate is any one or more of acrylic acid, malonic acid, esters of said acids, acrylates and glycols.

14. The E. coli strain of clam 1 wherein the glycerol 3-phosphatse has an amino acid sequence selected from the group consisting of SEQ ID NO:63 and SEQ ID NO:65
